(19) United States
(12) Patent Application Publication
(10) Pub. No.: US 2022/0265552 A1
(43) Pub. Date: Aug. 25, 2022

(54) ESLICARBAZEPINE SUSPENSION

(71) Applicant: Jubilant Generics Limited, Noida (IN)

(72) Inventors: Indranil Nandi, Yardley, PA (US); Dinesh Kumar, Noida (IN); Rakesh Singh, Noida (IN); TUSHARMOULI MUKHERJEE, Yardley, PA (US)

(73) Assignee: Jubilant Generics Limited, Noida (IN)

(21) Appl. No.: 17/734,420

(22) Filed: May 2, 2022

Related U.S. Application Data
(63) Continuation-in-part of application No. 17/327,203, filed on May 21, 2021, now Pat. No. 11,318,145.
Application No. 17/327,203 is a continuation-in-part of application No. 16/827,534, filed on Mar. 23, 2020.
Application No. 16/827,534 is a continuation-in-part of application No. PCT/IB2018/057400, filed on Sep. 25, 2018.

(30) Foreign Application Priority Data
Sep. 25, 2017 (IN) 201711033833

(51) Int. Cl. A61K 9/08; A61K 31/55; A61K 9/00; A61K 47/26; A61K 47/38; A61K 47/12; A61K 47/14

(52) U.S. Cl. CPC A61K 9/08; A61K 31/55; A61K 9/0095; A61K 47/14; A61K 47/38; A61K 47/12; A61K 47/26

(57) ABSTRACT

The present invention relates to oral ready to use liquid pharmaceutical compositions of eslicarbazepine. It also relates to the processes for the preparation of said liquid compositions. The present invention provides liquid compositions of eslicarbazepine with desired technical attributes such as release profile, viscosity, pH, stability, and acceptable organoleptic properties. The prepared compositions are useful in patients having difficulties in swallowing tablets and provide the physician with providing a more convenient and less cumbersome posology. The suspension compositions as per present invention are useful for the treatment of seizures, partial-onset seizures, epilepsy, neuropathic pain, migraine, fibromyalgia, trigeminal neuralgia, bipolar disorders, attention disorders, anxiety disorders, affective disorders, schizoaffective disorders, sensorimotor disorders, and vestibular disorders.

### FIELD OF THE INVENTION

The present invention relates to orally administered liquid pharmaceutical compositions of eslicarbazepine. The liquid compositions are in the form of a ready to use suspension and suspension powder for reconstitution. It also relates to the processes for the preparation of said liquid compositions.

### BACKGROUND OF THE INVENTION

Eslicarbazepine acetate is an anticonvulsant drug. It is chemically known as (S)-10-acetoxy-10, 11-dihydro-5H-dibenz [b,f]azepine-5-carboxamide.

Eslicarbazepine acetate is marketed in the United States as an immediate release tablet in 200 mg, 400 mg, 600 mg, and 800 mg strengths under the brand name APTIOM® (Eslicarbazepine acetate Oral Tablets 200 mg, 400 mg, 600 mg, and 800 mg) by Sunovion Pharmaceuticals. The marketed solid dosage form of eslicarbazepine is indicated for the treatment of partial-onset seizures.

The tablet dosage form of eslicarbazepine has a large tablet size and weight. Difficulty in swallowing large tablets and capsules (dysphagia) is a problem for many patients and can lead to a variety of adverse events as well as induce significant non-compliance with the prescribed treatment regimens. Adolescents, children, and the elderly are particularly vulnerable population groups that are more likely than adults to experience difficulty in swallowing large tablets or capsules. Liquid dosage forms are designed with an objective of minimizing swallowing difficulties and are likely to improve patient compliance by reducing dysphagia-related adverse events due to large tablet size and accordingly providing a more convenient and less cumbersome posology.

U.S. Publication No. 2013/0040939 discloses oral suspension formulations of eslicarbazepine acetate comprising xanthan gum as a suspending agent, polyoxyethylene stearate as a wetting agent, and saccharin sodium as a sweetener. Xanthan gum suffers from drawbacks such as microbial contamination and causes side effects such as allergic reactions, flatulence, bloating, and stomach upset. Sodium saccharin is not preferred as a sweetener as it has carcinogenic potential.

There exists an unmet need in the art to provide liquid compositions of eslicarbazepine acetate which are stable, provide ease of administration, dose adjustment, and enhanced patient compliance. The inventors of the present invention have prepared eslicarbazepine ready to use suspension and suspension powder for reconstitution convenient for administration by pediatric and geriatric patients, easy to manufacture, palatable, functionally reproducible, and provide ease of dose adjustment. Further, the liquid compositions exhibit desirable technical attributes.

### OBJECTS AND SUMMARY OF THE INVENTION

It is a principal object of the present invention to provide a stable immediate release oral liquid pharmaceutical composition comprising an anticonvulsant drug with one or more pharmaceutically acceptable excipients and processes for its preparation.

It is another object of the present invention to provide a pharmaceutical suspension comprising eslicarbazepine or its pharmaceutically acceptable esters, salts, solvates, polymorphs, enantiomers, or mixtures thereof with one or more pharmaceutically acceptable excipients and/or carrier and process for their preparation.

It is another object of the present invention to provide a suspension powder for reconstitution comprising eslicarbazepine or its pharmaceutically acceptable esters, salts, solvates, polymorphs, enantiomers, or mixtures thereof with one or more pharmaceutically acceptable excipients and/or carrier and process for their preparation.

It is another object of the present invention to provide an oral liquid pharmaceutical composition in the form of a suspension comprising eslicarbazepine or its pharmaceutically acceptable esters, salts, solvates, polymorphs, enantiomers, or mixtures thereof with one or more pharmaceutically acceptable excipients and/or carrier, wherein the pharmaceutically acceptable excipients are selected from the group comprising of suspending agent, antioxidant, anticaking agent, antifoaming agent, pH adjusting agent, coloring agent, sweetening agent, flavoring agent, surfactant/solubilizer/wetting agent, buffer, diluent, preservative, and mixtures thereof. The suspension is in the form of ready to use suspension and suspension powder for reconstitution.

The following embodiments further describe the objects of the present invention in accordance with the best mode of practice, however, the disclosed invention is not restricted to the particular embodiments hereinafter described.

In accordance with one embodiment of the present invention, there is provided an immediate release oral pharmaceutical suspension dosage form comprising:

a) eslicarbazepine or its pharmaceutically acceptable salts, esters, hydrates, or polymorphs thereof present at from about 0.1% to about 40% w/w;

b) suspending agent present at from about 0.01% to about 10% w/w; and

c) surfactant present at from 0% to about 7% w/w; wherein the dosage form is free from xanthan gum and/or polyoxyethylene stearate.

In accordance with another embodiment of the present invention, there is provided an immediate release oral pharmaceutical suspension dosage form comprising:

a) eslicarbazepine or its pharmaceutically acceptable salts, esters, hydrates or polymorphs thereof present at from about 0.1% to about 40% w/w;

b) suspending agent present at from about 0.01% to about 10% w/w; and

c) surfactant present at from 0% to about 7% w/w; wherein the pH of the suspension is from 3 to 8 and the dosage form is free from xanthan gum and/or polyoxyethylene stearate.

In accordance with another embodiment of the present invention, there is provided an immediate release oral pharmaceutical suspension dosage form comprising:

a) eslicarbazepine or its pharmaceutically acceptable salts, esters, hydrates, or polymorphs thereof present at from about 0.1% to about 40% w/w;

b) suspending agent present at from about 0.01% to about 10% w/w;

c) surfactant present at from 0% to about 7% w/w; and

d) pharmaceutically acceptable liquid carrier; wherein the dosage form is free from xanthan gum and/or polyoxyethylene stearate.

In accordance with another embodiment of the present invention, there is provided an immediate release oral pharmaceutical suspension dosage form comprising:

a) eslicarbazepine or its pharmaceutically acceptable salts, esters, hydrates, or polymorphs thereof present at from about 0.1% to about 40% w/w;

b) suspending agent present at from about 0.01% to about 10% w/w; and

c) surfactant present at from 0% to about 7% w/w;

wherein the dosage form is free from xanthan gum and/or polyoxyethylene stearate and exhibits more than 65% of drug release within 20 minutes, when placed in a dissolution vessel filled with 1000 ml of acetate buffer, pH 4.5 maintained at 37±0.5° C. and stirred at a paddle speed of 100 rpm using a USP Type II (paddle) apparatus.

In accordance with another embodiment of the present invention, the immediate release oral pharmaceutical suspension dosage form of eslicarbazepine is a ready to use suspension. In another embodiment, the immediate release oral pharmaceutical suspension dosage form of eslicarbazepine is a suspension powder for reconstitution.

In accordance with another embodiment of the present invention, the immediate release oral pharmaceutical suspension dosage form has eslicarbazepine in an amount from about 0.1 mg/mL to about 200 mg/mL and a viscosity of from about 700 cps to about 1200 cps.

In accordance with another embodiment of the present invention, there is provided an immediate release oral pharmaceutical suspension dosage form of eslicarbazepine or its pharmaceutically acceptable salts, esters, hydrates, or polymorphs thereof with one or more pharmaceutically acceptable excipients comprising:

a) suspending agent;

b) preservative;

c) optionally a surfactant;

d) optionally an antioxidant; and

e) pH adjusting agent to maintain the pH of the composition in the range of about 3 to about 8; and/or pharmaceutically acceptable liquid carrier; wherein the composition is free of xanthan gum and/or polyoxyethylene stearate.

In accordance with another embodiment of the present invention, there is provided an immediate release oral pharmaceutical suspension dosage form of eslicarbazepine or its pharmaceutically acceptable salts, esters, hydrates, or polymorphs thereof with one or more pharmaceutically acceptable excipients comprising:

a) suspending agent;

b) diluent;

c) preservative;

d) optionally an antioxidant; and

e) pH adjusting agent to maintain the pH of the composition in the range of about 3 to about 8; and/or pharmaceutically acceptable liquid carrier, wherein the suspending agent is selected from the group comprising gellan gum, cellulose, and its derivatives, a mixture of carboxymethylcellulose and microcrystalline cellulose, propylene glycol alginate, and combinations thereof.

In accordance with another embodiment of the present invention, there is provided an immediate release oral pharmaceutical suspension dosage form of eslicarbazepine or its pharmaceutically acceptable salts, esters, hydrates, or polymorphs thereof with one or more pharmaceutically acceptable excipients comprising:

a) a suspending agent;

b) a surfactant;

c) a preservative;

d) optionally an antioxidant; and

e) pH adjusting agent in sufficient amounts to maintain the pH of the composition in the range of about 3 to about 8; and/or pharmaceutically acceptable liquid carrier,

wherein the surfactant is selected from the group comprising non-ionic surfactants, anionic, cationic, or zwitterionic surfactants with the proviso that the surfactant is not polyoxyethylene stearate.

In accordance with another embodiment of the present invention, there is provided an immediate release oral pharmaceutical suspension dosage form of eslicarbazepine or its pharmaceutically acceptable salts, esters, hydrates or polymorphs thereof with one or more pharmaceutically acceptable excipients comprising eslicarbazepine present at from about 0.1 mg/mL to about 200 mg/mL, wherein the pH of the composition is from 3 to 8.

In accordance with another embodiment of the present invention, there is provided an immediate release oral pharmaceutical suspension dosage form of eslicarbazepine or its pharmaceutically acceptable salts, esters, hydrates or polymorphs thereof with one or more pharmaceutically acceptable excipients comprising eslicarbazepine present at from about 0.1 mg/mL to about 200 mg/mL, wherein the pH of the composition is from 3 to 8 and the viscosity is from 700 cps to 1200 cps.

In accordance with one embodiment of the present invention, there is provided a suspension powder for reconstitution comprising eslicarbazepine or its pharmaceutically acceptable esters, salts, hydrates, or polymorphs thereof and at least one or more pharmaceutically acceptable excipients selected from the group comprising suspending agent/thickening agent/viscosity agent, antioxidant, anticaking agent, antifoaming agent, pH adjusting agent, coloring agent, sweetening agent, flavoring agent, surfactant/wetting agent, buffer, diluent and preservative.

In accordance with one embodiment of the present invention, there is provided a ready to use stable liquid suspension comprising eslicarbazepine or its pharmaceutically acceptable esters, salts, esters, hydrates, or polymorphs thereof and at least one or more pharmaceutically acceptable excipients and/or carrier wherein the pharmaceutically acceptable excipient is selected from the group comprising a thickening agent/viscosity agent, antioxidant, anticaking agent, antifoaming agent, pH adjusting agent, coloring agent, sweetening agent, flavoring agent, surfactant/wetting agent, buffer, diluent, taste-masking agent, and preservative.

In accordance with another embodiment of the present invention, there is provided dry powder for suspension compositions suitable for use as a liquid suspension for children or elderly patients. The compositions include eslicarbazepine or its pharmaceutically acceptable salts, esters, hydrates, or polymorphs thereof and pharmaceutically acceptable excipients selected from the group consisting of suspending agents, coating agents, preservatives, flavoring agents, sweeteners, lubricants, surfactants, buffering agents, and diluents.

In accordance with still another embodiment of the present invention, there is provided a process for the preparation of a stable pharmaceutical composition in the form of a suspension comprising eslicarbazepine or its pharmaceutically acceptable salts, esters, hydrates, or polymorphs thereof.

In accordance with still another embodiment of the present invention, there is provided a process for the preparation of a stable pharmaceutical composition in the form of a suspension comprising eslicarbazepine or its pharmaceutically acceptable esters, salts, hydrates, or polymorphs thereof in micronized form.

In accordance with still another embodiment of the present invention, there is provided a process for the preparation of a ready to use liquid suspension of eslicarbazepine or its pharmaceutically acceptable salts, esters, hydrates, or polymorphs thereof, comprising combining various components using conventional equipment such as overhead stirrers, ultrasonifiers, mills, and homogenizers. Many different orders of adding components can be employed.

In accordance with still another embodiment of the present invention, there is provided a process for the preparation of a dry powder for suspension composition of eslicarbazepine or its pharmaceutically acceptable salts, esters, hydrates, or polymorphs thereof, which is suitable for suspension in water and/or water-miscible suitable solvents to form an orally administrable product which comprises admixing eslicarbazepine granules with substantially dry pharmaceutically acceptable excipients selected from the group consisting of suspending agents/viscosity enhancers, coating agents, preservatives, flavoring agents, sweeteners, lubricants, wetting agents, surfactants, buffering agents, and diluents to form a dry admixture, and transferring the dry admixture to a sealable storage container.

In accordance with still another embodiment of the present invention, there is provided a stable pharmaceutical composition in the form of a suspension comprising eslicarbazepine or its pharmaceutically acceptable salts, esters, hydrates, or polymorphs thereof, wherein the composition is substantially free from other polymorphic forms.

In accordance with still another embodiment of the present invention, there is provided a stable pharmaceutical composition in the form of a suspension comprising eslicarbazepine or its pharmaceutically acceptable salts, esters, hydrates, or polymorphs thereof, wherein eslicarbazepine has a particle size distribution D90 less than about 200 μm.

In accordance with still another embodiment of the present invention, there is provided a stable pharmaceutical composition in the form of a suspension comprising eslicarbazepine or its pharmaceutically acceptable salts, esters, hydrates, or polymorphs thereof, in an amount of about 0.01% to about 90% by weight, wherein the composition exhibits desirable technical attributes like pourability, viscosity, dissolution, stability, re-suspendability, and re-dispersibility.

In accordance with still another embodiment of the present invention, there is provided a use of a stable pharmaceutical composition in the form of a suspension comprising eslicarbazepine or its pharmaceutically acceptable salts, hydrates, or polymorphs, thereof, for the treatment of epilepsy, neuropathic pain, migraine, fibromyalgia, trigeminal neuralgia, bipolar disorders, attention disorders, anxiety disorders, affective disorders, schizoaffective disorders, sensorimotor disorders, and vestibular disorders.

In accordance with still another embodiment of the present invention, there is provided a kit comprising: (a) an immediate release oral pharmaceutical suspension dosage form comprising eslicarbazepine or its pharmaceutically acceptable salts, esters, hydrates, or polymorphs thereof with one or more pharmaceutically acceptable excipients and/or carrier, (b) a dispensing and/or dosing syringe for administering the composition; and (c) optionally, instructions for preparation and use.

### BRIEF DESCRIPTION OF THE FIGURE

FIG. 1 shows an overlay of the x-ray diffraction patterns of eslicarbazepine acetate drug substance and eslicarbazepine suspension (initial) and eslicarbazepine suspension stability samples (stored at 80° C. for 5 days) as per Example 11.

### DETAILED DESCRIPTION OF THE INVENTION

The present invention can be more readily understood by the following detailed description of the invention and study of the included examples.

As used herein, the term “composition” or “formulation” or “dosage form”, as in pharmaceutical composition, is intended to encompass a drug product comprising an anticonvulsant or anti-epileptic drug, preferably eslicarbazepine or its pharmaceutically acceptable salts, esters, solvates, polymorphs, enantiomers or mixtures thereof, and other inert ingredient(s) (pharmaceutically acceptable excipients). Such pharmaceutical compositions are synonymous with “formulation” and “dosage form”. The pharmaceutical compositions of the present invention include, but are not limited to powder for suspension, ready to use suspension, and the like.

As used herein, the term “ready to use suspension” means a pre-constituted suspension which can be administered as such. The “powder for suspension” or “dry suspension” needs to be reconstituted with a liquid carrier to form a suspension before administration.

As used herein, the term “eslicarbazepine” is used in a broad sense to include not only “eslicarbazepine” per se but also its pharmaceutically acceptable salts, solvates, hydrates, enantiomers, derivatives, isomers, polymorphs, metabolites, prodrugs thereof, and also its various crystalline and amorphous forms. In particular, the salt of eslicarbazepine is eslicarbazepine acetate. The term “eslicarbazepine acetate” used in this specification means the S-isomer in substantially pure form, i.e. at least about 98% pure.

The term “excipient” means a pharmacologically inactive component such as a suspending agent/viscosity agent, anticaking agent, antifoaming agent, pH adjusting agent, antioxidant, sweetening agent, flavoring agent, surfactant/solubilizer/wetting agent, buffer, and preservative and the like. The excipients used in preparing the liquid pharmaceutical composition are safe and non-toxic. Reference to an excipient includes both one and more than one such excipient. Co-processed excipients are also covered under the scope of the present invention. Combinations of excipients performing the same function may also be used to achieve desired formulation characteristics.

As used herein, the term “about” means±approximately 20% of the indicated value, such that “about 10 percent” indicates approximately 08 to 12 percent.

The term “patient” and/or “subject” are used interchangeably herein. In some embodiments, the patient or subject is a human. In further embodiments, the patient or subject is an animal. In some embodiments, the human can be of any age such as adult, adolescent, pediatric or geriatric.

As used herein, the term “therapeutic agent” means an agent or drug utilized to treat, combat, ameliorate, prevent or improve an unwanted condition or allergy or disease, or infection of a patient. The composition may be administered, as the case may be, before or after the onset of an infection or disease, like for prophylactic or therapeutic treatment purposes, or both. The term “therapeutically equivalent” means a formulation where its therapeutic effect is equivalent to a reference formulation.

“Reference drug product” means an eslicarbazepine product as described in U.S. Federal Food and Drug Administration's New Drug Application No. 022416 as provided in the U.S. Federal Food and Drug Administration's Orange Book, Approved Drug Products with Therapeutic Equivalence Evaluations.

Bioavailability refers to the proportion of the drug administered that reaches the physiological site where the drug exerts its therapeutic effect, which is generally regarded as the bloodstream for many drugs. The bioavailability of a drug is most readily expressed as the concentration of the drug or its active metabolites in the blood plasma integrated over time. This quantity is commonly referred to as the “area under the curve” or “AUC”. Bioequivalence study: As per the USFDA guideline titled “Bioavailability and Bioequivalence Studies for Orally Administered Drug Products—General Considerations” Bioequivalence is defined as: “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.”

Tₘₐₓ refers to the observed time to reach a maximum plasma concentration. Cₘₐₓ refers to the maximum plasma concentration. Cₘᵢₙ refers to the minimum plasma concentration. AUC_{0-inf} refers to the area under the curve in a plot of analyte concentration in blood plasma versus time from zero to infinity. AUC₀₋ₜ refers to the area under the curve in a plot of analyte concentration in blood plasma versus time from zero to a specified time. AUC₀₋ₜₐᵤ refers to the area under the concentration-time curve from time 0 to the time of the last quantifiable concentration. T_{1/2} refers to the time required to eliminate one-half of the plasma concentration of an analyte. Kel refers to the elimination rate constant.

As used in this specification, the singular forms “a”, “an”, and “the” include plural references unless the context clearly dictates otherwise. Thus, for example, a reference to “a process” includes one or more processes, and/or steps of the type described herein and/or which will become apparent to those persons skilled in the art upon reading this disclosure and so forth.

The term “stable,” as used herein, refers to chemical stability, wherein not more than 5% w/w of total related substances are formed on storage at 40° C. and 75% relative humidity (R.H.) or at 25° C. and 60% R.H. for a period of at least one month, particularly for a period of two months, and more particularly for a period of at least three months.

The term “sedimentation volume ratio,” or “sedimentation ratio,” refers to a ratio of the ultimate volume of sediment (Vu) to the original volume of sediment (VO) before settling. It also refers to a ratio of the ultimate height of sediment (Hu) to the initial sediment (HO) before settling.

The term “taste-masking agents”, when used herein, refers to taste receptor blockers, compounds that mask the chalkiness, grittiness, dryness, and/or astringent or bitter taste properties of an active compound.

Unless otherwise stated the weight percentages expressed herein are based on the final weight of the composition or formulation.

The present invention is a stable pharmaceutical composition directed to ready to use oral liquid suspension or dry powder for suspension compositions suitable for use as a liquid suspension for administration to a subject in need thereof which comprises eslicarbazepine or its pharmaceutically acceptable salts, esters, hydrates or polymorphs thereof.

In accordance with one embodiment of the present invention, there is provided a stable pharmaceutical composition in the form of a suspension comprising eslicarbazepine or its pharmaceutically acceptable salts, esters, hydrates, or polymorphs thereof with one or more pharmaceutically acceptable excipient and/or liquid carrier and process for its preparation.

Another embodiment of the present invention relates to an immediate release oral pharmaceutical suspension dosage form of eslicarbazepine or its pharmaceutically acceptable salts, esters, hydrates, or polymorphs thereof with one or more pharmaceutically acceptable excipients comprising:

a) suspending agent;

b) preservative;

c) optionally a surfactant;

d) optionally an antioxidant; and

e) pH adjusting agent to maintain the pH of the composition in the range of about 3 to about 8; and/or pharmaceutically acceptable liquid carrier.

In accordance with another embodiment of the present invention, there is provided a suspension comprising eslicarbazepine or its pharmaceutically acceptable salts, esters, hydrates, or polymorphs thereof and one or more pharmaceutically acceptable excipients and/or a liquid carrier, wherein the composition is free of xanthan gum.

In accordance with another embodiment of the present invention, there is provided a suspension comprising eslicarbazepine or its pharmaceutically acceptable salts, esters, hydrates, or polymorphs thereof and one or more pharmaceutically acceptable excipients and/or a liquid carrier, wherein the composition is free of polyoxyethylene stearate.

In accordance with another embodiment of the present invention, there is provided a stable suspension comprising eslicarbazepine or its pharmaceutically acceptable salts, esters, hydrates, or polymorphs thereof and one or more pharmaceutically acceptable excipients and/or a liquid carrier, wherein the composition is free of xanthan gum and polyoxyethylene stearate.

In accordance with another embodiment of the present invention, there is provided an immediate release oral ready to use suspension comprising:

a) eslicarbazepine or its pharmaceutically acceptable salts, esters, hydrates, or polymorphs thereof at from about 0.1% to about 40% w/w;

b) suspending agent at from about 0.01% to about 10% w/w;

c) surfactant at from about 0.01% to about 7% w/w; and

d) pharmaceutically acceptable liquid carrier at from about 10% to about 95% w/w; wherein the suspension is free from xanthan gum and polyoxyethylene stearate.

In accordance with another embodiment of the present invention, there is provided an immediate release oral ready to use suspension of comprising:

a) eslicarbazepine or its pharmaceutically acceptable salts, esters, hydrates, or polymorphs thereof at from about 0.1% to about 40% w/w;

b) a suspending agent at from about 0.01% to about 10% w/w;

c) a surfactant from at about 0.01% to about 7% w/w; and

d) a pharmaceutically acceptable liquid carrier at from about 10% to about 95% w/w; wherein the ratio of eslicarbazepine: suspending agent ranges from 1:0.01 to 1:4 by weight and the suspension is free of xanthan gum and polyoxyethylene stearate.

In accordance with another embodiment of the present invention, there is provided an immediate release oral ready to use suspension, wherein the suspension comprises:

a) eslicarbazepine or its pharmaceutically acceptable salts, esters, hydrates, or polymorphs thereof at from about 0.1% to about 40% w/w;

b) a suspending agent at from about 0.01% to about 10% w/w;

c) a surfactant at from about 0.01% to about 7% w/w; and

d) a pharmaceutically acceptable liquid carrier at from about 10% to about 95% w/w; wherein the pH of the suspension is from 3 to 6.5 and the suspension is free of xanthan gum and polyoxyethylene stearate.

In accordance with another embodiment of the present invention, there is provided an immediate release oral ready to use suspension, wherein there is no change in the polymorphic form of eslicarbazepine for at least 5 days when the suspension is stored at 80° C.

In accordance with another embodiment of the present invention, there is provided an immediate release oral ready to use suspension, wherein the suspension is stable when stored at 40° C./75% R.H. for a period of at least 3 months.

In accordance with another embodiment of the present invention, there is provided an immediate release oral ready to use suspension, wherein the viscosity of the suspension is from 100 to 5000 cps.

In accordance with another embodiment of the present invention, there is provided an immediate release oral ready to use suspension, wherein the viscosity of the suspension is from 100 to 2500 cps.

In accordance with another embodiment of the present invention, there is provided an immediate release oral ready to use suspension, wherein the amount of eslicarbazepine is from about 0.1 mg/mL to about 400 mg/mL.

In accordance with another embodiment of the present invention, there is provided an immediate release oral ready to use suspension comprising:

a) eslicarbazepine or its pharmaceutically acceptable salts, esters, hydrates, or polymorphs thereof;

b) suspending agent;

c) surfactant;

d) preservative;

e) pH adjusting agent or buffering agent;

f) sweetening agent; and

h) pharmaceutically acceptable liquid carrier;

wherein the suspension is free from xanthan gum and polyoxyethylene stearate.

In accordance with another embodiment of the present invention, there is provided an immediate release oral ready to use suspension comprising:

a) eslicarbazepine or its pharmaceutically acceptable salts, esters, hydrates, or polymorphs thereof;

b) co-processed spray-dried forms of microcrystalline cellulose and carboxymethyl cellulose sodium as suspending agent;

c) surfactant;

d) preservative;

e) pH adjusting agent or buffering agent;

f) sweetening agent;

g) flavoring agent; and

h) pharmaceutically acceptable liquid carrier;

wherein the suspension is free from xanthan gum and polyoxyethylene stearate.

In accordance with another embodiment of the present invention, there is provided an immediate release oral ready to use suspension comprising:

a) eslicarbazepine or its pharmaceutically acceptable salts, esters, hydrates, or polymorphs thereof;

b) one or more suspending agents selected from cellulose derivatives and co-processed spray-dried forms of microcrystalline cellulose and carboxymethyl cellulose sodium, carbomers, gums, pectin, propylene glycol alginate, dextran, gelatin, polyethylene glycols, polyvinyl compounds, sugar alcohols, colloidal silica, maltodextrin, starch, or a combination thereof;

c) surfactant;

d) preservative;

e) pH adjusting agent or buffering agent;

f) sweetening agent;

g) flavoring agent; and

h) pharmaceutically acceptable liquid carrier;

wherein the suspension is free from xanthan gum and polyoxyethylene stearate.

In accordance with another embodiment of the present invention, there is provided an immediate release oral ready to use suspension comprising:

a) eslicarbazepine or its pharmaceutically acceptable salts, esters, hydrates, or polymorphs thereof at from about 0.1% to about 40% w/w;

b) suspending agent at from about 0.01% to about 10% w/w;

c) surfactant at from about 0.01% to about 7% w/w;

d) preservative at from about 0.001% to about 4% w/w;

e) pH adjusting agent or buffering agent at from about 0.01% to about 15% w/w; f) sweetening agent at from about 0.01% to about 70% w/w;

g) flavoring agent at from about 0.01% to about 5% w/w; and

h) pharmaceutically acceptable liquid carrier at from about 10% to about 95% w/w; wherein the suspension is free from xanthan gum and polyoxyethylene stearate.

In accordance with another embodiment of the present invention, there is provided an immediate release oral ready to use suspension comprising:

a) eslicarbazepine or its pharmaceutically acceptable salts, esters, hydrates, or polymorphs thereof;

b) co-processed spray-dried forms of microcrystalline cellulose and carboxymethyl cellulose sodium or a combination thereof;

c) polyoxyethylene sorbitan fatty acid ester surfactant;

d) paraben;

e) citric acid and trisodium citrate buffer;

f) sorbitol or thaumatin or a combination thereof;

g) flavoring agent;

h) glycerin; and

i) water, wherein the suspension is free from xanthan gum and polyoxyethylene stearate.

In accordance with another embodiment of the present invention, there is provided an immediate release oral ready to use suspension comprising:

a) eslicarbazepine or its pharmaceutically acceptable salts, esters, hydrates, or polymorphs thereof at from about 0.1% to about 40% w/w;

b) co-processed spray-dried forms of microcrystalline cellulose and carboxymethyl cellulose sodium, or a combination thereof at from about 0.01% to about 10% w/w;

c) polyoxyethylene sorbitan fatty acid ester surfactant at from about 0.01% to about 7% w/w;

d) paraben at from about 0.001% to about 3% w/w;

e) citric acid and trisodium citrate buffer at from about 0.005% to about 4% w/w;

f) sorbitol or thaumatin or a combination thereof at from about 0.01% to about 70% w/w;

g) flavoring agent at from about 0.01% to about 5% w/w;

h) glycerin, and

i) water, wherein the suspension is free from xanthan gum and polyoxyethylene stearate.

In accordance with another aspect of the present embodiment, there is provided an immediate release oral pharmaceutical ready to use suspension comprising:

(a) eslicarbazepine or its pharmaceutically acceptable salts, esters, hydrates, or polymorphs thereof at from about 0.1% to about 40% w/w;

(b) suspending agent at from about 0.01% to about 10% w/w;

(c) surfactant at from about 0.01% to about 7% w/w; and

(d) pharmaceutically acceptable liquid carrier at from about 10% to about 95% w/w; wherein the suspension is free of xanthan gum and polyoxyethylene stearate and exhibits more than 75% of drug release within 15 minutes, when placed in a dissolution vessel filled with 1000 ml of acetate buffer, pH 4.5 maintained at 37±0.5° C. and stirred at a paddle speed of 100 rpm using a USP Type II (paddle) apparatus.

In accordance with another embodiment of the present invention, there is provided an immediate release oral ready to use suspension comprising:

a) eslicarbazepine or its pharmaceutically acceptable salts, esters, hydrates, or polymorphs thereof at from about 0.1% to about 40% w/w;

b) a suspending agent at from about 0.01% to about 10% w/w;

c) a surfactant at from about 0.01% to about 7% w/w;

d) a preservative at from about 0.001% to about 3% w/w; and

e) a pharmaceutically acceptable liquid carrier at from about 10% to about 95% w/w, wherein the suspension is free from xanthan gum and polyoxyethylene stearate.

In accordance with another embodiment of the present invention, there is provided an immediate release oral ready to use suspension for use in the treatment of seizures, partial-onset seizures, epilepsy, neuropathic pain, migraine, fibromyalgia, trigeminal neuralgia, bipolar disorders, attention disorders, anxiety disorders, affective disorders, schizoaffective disorders, sensorimotor disorders, and vestibular disorders.

In accordance with another aspect of the present embodiment, there is provided a stable suspension comprising eslicarbazepine or its pharmaceutically acceptable salts, wherein the suspending agent is selected from gellan gum, sodium carboxymethylcellulose, a mixture of carboxymethylcellulose and microcrystalline cellulose, propylene glycol alginate and combinations thereof.

In accordance with another embodiment of the present invention, there is provided an immediate release oral ready to use suspension comprising (a) eslicarbazepine or its pharmaceutically acceptable salts, esters, hydrates, or polymorphs thereof and (b) suspending agent, wherein the suspending agent is effective for maintaining a sedimentation volume ratio of more than about 0.8 for at least 10 hours after the suspension is prepared. In accordance with another embodiment of the present invention, the suspension maintains a sedimentation volume ratio of more than about 0.7, more than about 0.8, or more than about 0.9 for a period of at least about 10 hours, at least about 12 hours, at least about 15 hours, at least about 18 hours, at least about 20 hours, at least 1 day, at least 2 days, or at least 6 days after the suspension is prepared.

In accordance with another embodiment of the present invention, there is provided an immediate release oral ready to use suspension comprising (a) eslicarbazepine or its pharmaceutically acceptable salts, esters, hydrates, or polymorphs thereof and (b) a suspending agent, wherein the ratio of eslicarbazepine: suspending agent ranges from 1:0.01 to 1:4 by weight. In another embodiment, the ratio of eslicarbazepine: suspending agent ranges from 1:0.05 to 1:1 by weight. In yet another embodiment, the ratio of eslicarbazepine: suspending agent ranges from 1:0.05 to 1:0.5 by weight.

In accordance with another embodiment of the present invention, there is provided an immediate release oral ready to use suspension comprising eslicarbazepine or its pharmaceutically acceptable salts, esters, hydrates, or polymorphs thereof, wherein the suspension is bioequivalent to the marketed eslicarbazepine tablet (APTIOM®—Eslicarbazepine acetate Oral Tablets 200 mg, 400 mg, 600 mg, and 800 mg).

In accordance with another embodiment of the present invention, there is provided an immediate release oral pharmaceutical ready to use suspension comprising eslicarbazepine or its pharmaceutically acceptable salts, esters, hydrates, or polymorphs thereof, wherein the suspension exhibits a comparable dissolution compared to the commercially marketed tablet of eslicarbazepine (APTIOM®—Eslicarbazepine acetate Oral Tablets 200 mg, 400 mg, 600 mg, and 800 mg).

In accordance with another embodiment of the present invention, there is provided an immediate release oral pharmaceutical ready to use suspension comprising eslicarbazepine or its pharmaceutically acceptable salts, esters, hydrates, or polymorphs thereof, in which the bitter taste of eslicarbazepine is masked by one or more taste-masking agents.

In accordance with another embodiment of the present invention, there is provided an immediate release oral pharmaceutical ready to use suspension comprising eslicarbazepine or its pharmaceutically acceptable salts, esters, hydrates, or polymorphs thereof, wherein the suspension is free of propylene glycol.

In accordance with another embodiment of the present invention, sucrose has a particle size such that not less than 90% of particles are below 200 μm. In particular, sucrose has a particle size such that not less than 90% of particles are below 100 μm. This helps in achieving improved uniformity of the drug in the mixture.

In accordance with other embodiment of the present invention, there is provided a stable suspension comprising about 0.01% to about 90% by weight of eslicarbazepine or its pharmaceutically acceptable salts, esters, hydrates, or polymorphs thereof, preferably in the range of about 0.1% to about 40% by weight on the basis of the total weight of the composition.

In accordance with other embodiment of the present invention, there is provided a stable suspension comprising eslicarbazepine or its pharmaceutically acceptable salts, esters, hydrates, or polymorphs thereof, wherein the amount of eslicarbazepine in the suspension ranges from about 0.1 mg/mL to about 400 mg/mL. The amount of eslicarbazepine in the suspension ranges preferably from about 0.5 mg/mL to 300 mg/mL, preferably from about 0.5 mg/mL to 200 mg/mL, preferably from about 0.5 mg/mL to 100 mg/mL. More preferably the amount of eslicarbazepine in the suspension ranges from about 0.5 mg/mL to 75 mg/mL.

In accordance with one aspect of the present embodiment, there is provided a stable suspension comprising eslicarbazepine or its pharmaceutically acceptable salts, esters, hydrates or polymorphs thereof, wherein the amount of eslicarbazepine in suspension is 1 mg/mL, 2 mg/5 mL, 5 mg/5 mL, 25 mg/5 mL, 50 mg/5 mL, 100 mg/5 mL and 250 mg/5 mL, 5 mg/mL, 25 mg/mL, 50 mg/mL, 100 mg/mL and 250 mg/mL. Particularly, the amount of eslicarbazepine in suspension is 50 mg/mL and 100 mg/mL.

In accordance with other embodiment of the present invention, there is provided a stable suspension comprising eslicarbazepine or its pharmaceutically acceptable salts, esters, hydrates, or polymorphs thereof, wherein the pH of the suspension is in range of 2 to 9. Preferably, the pH of the suspension is in a range of 2 to 8, and in a range of 3 to 8. More preferably, the pH of the suspension is in a range of 3.0 to 6.5 and in a range of 3.5 to 6.5. More preferably, the pH of the suspension is in a range of 3.0 to 5.5. In accordance with other embodiment of the present invention, there is provided a stable suspension comprising eslicarbazepine or its pharmaceutically acceptable salts, esters, hydrates, or polymorphs thereof, wherein the suspension is a liquid suspension packaged in a bottle.

In accordance with yet another embodiment of the present invention, there is provided a stable suspension comprising eslicarbazepine or its pharmaceutically acceptable salts, esters, hydrates, or polymorphs thereof, wherein the suspension is a powder for suspension packaged in a bottle or sachet.

In accordance with yet another embodiment of the present invention, there is provided a suspension comprising eslicarbazepine or its pharmaceutically acceptable salts, esters, hydrates, or polymorphs thereof, wherein the suspension is free of any sugar.

In accordance with yet another embodiment of the present invention, there is provided a suspension comprising eslicarbazepine or its pharmaceutically acceptable salts, esters, hydrates, or polymorphs thereof, wherein the suspension is free of any dye.

In accordance with yet another embodiment of the present invention, there is provided a suspension comprising eslicarbazepine or its pharmaceutically acceptable salts, esters, hydrates, or polymorphs thereof, wherein the suspension is free of any natural gum.

In accordance with yet another embodiment of the present invention, there is provided a suspension comprising eslicarbazepine or its pharmaceutically acceptable salts, esters, hydrates, or polymorphs thereof, wherein the suspension is free of saccharin sodium.

In accordance with yet another embodiment of the present invention, there is provided a suspension comprising eslicarbazepine or its pharmaceutically acceptable salts, esters, hydrates, or polymorphs thereof, wherein the suspension is free of xanthan gum, polyoxyethylene stearate, and saccharin sodium.

In accordance with yet another embodiment of the present invention, there is provided a suspension comprising eslicarbazepine or its pharmaceutically acceptable salts, esters, hydrates, or polymorphs thereof, wherein the suspension is administered at least once daily.

In accordance with yet another embodiment of the present invention, there is provided a suspension comprising eslicarbazepine or its pharmaceutically acceptable salts, esters, hydrates, or polymorphs thereof, wherein the suspension contains bubble gum flavor, peppermint flavor, fantasy fruit masking flavor, strawberry flavor, or their combination as the flavoring agents.

In accordance with yet another embodiment of the present invention, there is provided a suspension comprising eslicarbazepine acetate, wherein the total impurities/related substances are not more than 2% when the suspension is stored at 40° C./75% R.H. for a period of at least 3 months.

In accordance with yet another embodiment of the present invention, there is provided a suspension comprising eslicarbazepine or its pharmaceutically acceptable salts, esters, hydrates, or polymorphs thereof, wherein the suspension is provided in a kit comprising:

(a) an immediate release oral pharmaceutical ready to use suspension comprising eslicarbazepine; and

(b) a dosing syringe with a colored plunger and clear barrel for administering the suspension.

In accordance with yet another embodiment of the present invention, there is provided a suspension comprising eslicarbazepine or its pharmaceutically acceptable salts, esters, hydrates, or polymorphs thereof, wherein the suspension is provided in a kit comprising:

(a) an immediate release oral pharmaceutical ready to use suspension comprising eslicarbazepine; and

(b) a dosing syringe with a colored plunger and clear barrel for administering the suspension, wherein the suspension is free from xanthan gum and polyoxyethylene stearate.

According to another embodiment of the present invention, eslicarbazepine has a particle size distribution D90 less than about 200 μm. Eslicarbazepine has a particle size distribution D90 between 5 μm and 200 μm. Eslicarbazepine has a particle size distribution particularly D90 between 5 μm and 175 μm, particularly D90 between 5 μm and 150 μm, particularly D90 between 5 μm and 125 μm, particularly D90 between 5 μm and 100 μm, particularly D90 between 5 μm and 75 μm, and particularly D90 between 5 μm and 50 μm.

In accordance with one embodiment of the present invention, there is provided a process for the preparation of a stable suspension comprising eslicarbazepine or its pharmaceutically acceptable salts, esters, hydrates, or polymorphs thereof, and one or more pharmaceutically acceptable excipients and/or a liquid carrier, wherein the process utilized is blending, dry granulation, wet granulation, spheronization extrusion process, hot melt extrusion process, homogenization or the like.

In accordance with one embodiment of the present invention, there is provided a process for the preparation of a ready to use suspension, wherein the process comprises the following steps:

(i) dissolving/dispersing one or more pharmaceutically acceptable excipients in a portion of water;

(ii) dispersing eslicarbazepine or its pharmaceutically acceptable salts, esters, hydrates, or polymorphs thereof in the solution/dispersion of step (i) to form a dispersion;

(iii) mixing suspending agent in another portion of water;

(iv) adding the mixture of step (iii) to the dispersion of step (ii);

(v) optionally adding one or more pharmaceutically acceptable excipients to the dispersion of step (iv); and

(vi) optionally homogenizing the mixture of step (iv) to form a suspension.

In accordance with one embodiment of the present invention, there is provided a process for preparation of a ready to use suspension, wherein the process comprises the following steps:

(i) mixing suspending agent, pH adjusting agent/buffering agent, sweetener, and preservative in a portion of a liquid carrier to form a dispersion;

(ii) mixing eslicarbazepine or its pharmaceutically acceptable salts, esters, hydrates, or polymorphs thereof and surfactant in another portion of a liquid carrier to form a dispersion;

(iii) adding the dispersion of step (ii) to the dispersion of step (i); and

(iv) adding one or more pharmaceutically acceptable excipients (sweetener, flavoring agent) to the dispersion of step (iii) and the remaining portion of the liquid carrier to form a suspension.

In accordance with other embodiment of the present invention, there is provided a process for preparation of a powder for suspension, wherein process comprises the following steps:

(i) mixing eslicarbazepine or its pharmaceutically acceptable salts, esters, hydrates, or polymorphs thereof with one or more pharmaceutically acceptable excipients;

(ii) granulating the mixture of step (i) using a solvent;

(iii) drying the granulated mixture of step (ii);

(iv) milling the mixture of step (iii) to form granules; and

(v) mixing the granules of step (iv) optionally with one or pharmaceutically acceptable excipients to form the suspension powder for reconstitution.

In accordance with other embodiment of the present invention, there is provided a process for the preparation of a suspension powder for reconstitution, wherein process comprises the following steps:

(i) mixing eslicarbazepine or its pharmaceutically acceptable salts, esters, hydrates, or polymorphs thereof with one or more pharmaceutically acceptable excipients;

(ii) compacting the mixture of step (i) to form slugs;

(iii) milling the slugs of step (ii) to form granules; and

(v) mixing the granules of step (iii) optionally with one or pharmaceutically acceptable excipients to form the suspension powder for reconstitution.

In accordance with other embodiment of the present invention, there is provided a process for the preparation of a suspension powder for reconstitution, wherein process comprises the following steps:

(i) mixing eslicarbazepine or its pharmaceutically acceptable salts, esters, hydrates, or polymorphs thereof with one or more pharmaceutically acceptable excipients; and

(ii) optionally lubricating the mixture of step (i) to form the suspension powder for reconstitution.

Powder/granules for oral suspension can be reconstituted using water or powder/granules for oral suspension can be administered by sprinkling the powder/granules on one teaspoonful of apple sauce or empty granules into a small cup or teaspoon containing one teaspoon of apple juice.

The suspension of the present invention provides advantages such as absence of lumps even after long storage when the composition is shaken as well as good pourability. The suspension of the invention has good physical stability properties such as low level of sedimentation (reduced or no caking) and easy re-dispersion on agitation. Moreover, it provides dose uniformity during each administration.

In accordance with another embodiment of the present invention, there is provided a ready to use liquid suspension comprising eslicarbazepine or its pharmaceutically acceptable, salts, esters hydrates or polymorphs thereof and at least one or more pharmaceutically acceptable excipient and/or a liquid carrier comprising suspending agent, antioxidant, anticaking agent, antifoaming agent, pH adjusting agent, sweetening agent, flavoring agent, surfactant/wetting agent, buffer, and preservative wherein, the suspension is easily dispersible or re-suspendable in a pharmaceutically acceptable liquid carrier including aqueous and/or non-aqueous carrier.

In accordance with still another embodiment of the present invention, there is provided a stable suspension comprising eslicarbazepine or its pharmaceutically acceptable salts, esters, hydrates, or polymorphs thereof, wherein the composition is substantially free from other polymorphic forms.

In accordance with still another embodiment of the present invention, there is provided a suspension comprising eslicarbazepine or its pharmaceutically acceptable salts, esters, hydrates, or polymorphs thereof in an amount of about 0.01% to about 90% by weight wherein, the composition exhibits desirable technical attributes like pourability, viscosity, dissolution, stability, re-suspendability, and re-dispersibility.

In accordance with still another embodiment of the present invention, there is provided a kit comprising:

(a) an immediate release oral pharmaceutical suspension dosage form comprising eslicarbazepine or its pharmaceutically acceptable salts, esters, hydrates, or polymorphs thereof with one or more pharmaceutically acceptable excipients and/or carrier,

(b) a dispensing and/or dosing syringe for administering the composition, and

(c) optionally, instructions for preparation and use.

In accordance with still another embodiment of the present invention, there is provided a kit comprising:

(a) an immediate release oral pharmaceutical suspension dosage form comprising eslicarbazepine or its pharmaceutically acceptable salts, esters, hydrates, or polymorphs thereof with one or more pharmaceutically acceptable excipients and/or carrier,

(b) a dispensing and/or dosing syringe or a measuring cup for administering the composition, and

(c) optionally, instructions for preparation and use.

In accordance with still another embodiment of the present invention, there is provided a kit comprising:

(a) an immediate release oral pharmaceutical suspension dosage form comprising eslicarbazepine or its pharmaceutically acceptable salts, esters, hydrates, or polymorphs thereof with one or more pharmaceutically acceptable excipients and/or carrier,

(b) a measuring cup for administering the composition, and

(c) optionally, instructions for preparation and use.

In another embodiment, the liquid composition of the present invention includes particle size of eslicarbazepine or its pharmaceutically acceptable esters, salts, solvates, polymorphs, enantiomers, or mixtures thereof, having a particle size distribution such that D90 is less than about 200 μm, D50 is less than about 100 μm and D10 is less than about 50 μm. Particularly, D50 is between about 5 μm to about 100 μm. The particle size of eslicarbazepine can be measured by suitable techniques such as Laser light scattering (e.g. Malvern Light Scattering), Coulter counter, microscopy, Fraunhofer diffraction, and any other technique known in the art.

The present inventors have surprisingly found that there is an effect of particle size of the drug substance on the viscosity of the suspension compositions. The suspension compositions wherein the particle size of drug substance D90 is in the range of 5 to 50 μm were only able to achieve desired viscosity for the final suspension compositions. Contrary, suspension compositions wherein the particle size of drug substance D90 was lower or higher than this range failed to achieve the desired viscosity, i.e., viscosity achieved was less than 100 or more than 500 cps.

In another embodiment of the present invention, there is provided a suspension powder for reconstitution comprising eslicarbazepine or its pharmaceutically acceptable esters, salts, solvates, polymorphs, enantiomers, or mixtures thereof, present in an amount of more than 0.01% by weight based on the total weight of the composition with one or more pharmaceutically acceptable excipient and/or a liquid carrier such as suspending agent, antioxidant, anticaking agent, antifoaming agent, pH adjusting agent, sweetening agent, flavoring agent, solubilizer/wetting agent, buffer, preservative, aqueous or non-aqueous carrier and the like.

The buffer concentration has been found herein to affect the stability of the suspension composition and there is a significant effect of buffer concentration on the stability of the suspension composition prepared as per the present invention. The present inventors surprisingly found that increasing the buffer concentration from about 1 mM to 20 mM increased the stability profile of suspension compositions. Compositions comprising lower buffer concentrations were less stable, in particular when the buffer concentration was below 5 mM.

In other embodiments, the buffer concentration is about 5 mM to about 25 mM. In one embodiment, the buffer concentration is about 20 mM. In one embodiment, the buffer concentration is about 10 mM. In one embodiment, the suspension composition of the present invention having buffer concentration from about 5 mM to 25 mM is substantially free from any degradation and has stable pH and viscosity profile. In one embodiment, the suspension composition of the present invention having buffer concentration from about 5 mM to 20 mM is substantially free from any degradation and has stable pH and viscosity profile. In one preferred embodiment, the suspension composition of the present invention having buffer concentration from about 10 mM to 20 mM is substantially free from any degradation and has stable pH and viscosity profile. In one embodiment, the suspension composition of the present invention having buffer concentration from about 10 mM to 20 mM have less than 1% of total impurities. The particular buffer and buffer concentration of a given composition for long-term storage provided herein may be determined empirically using standard stability assays well known to those of skill in the art (see, e.g., the examples).

In one embodiment, the suspension composition of the present invention comprises less than 2% of total impurities. In one embodiment, the suspension composition of the present invention comprises less than 1% of SLB-1 impurity also known as (10S)-10-Hydroxy-10,11-dihydro-5H-dibenzo[b,f] azepine-5-carboxamide. In one embodiment, the suspension composition of the present invention comprises less than 1% of dehydro SLB impurity also known as 10-Acetoxy-5H-dibenzo[b,f] azepine-5-carboxamide. In one embodiment, the suspension composition of the present invention comprises less than 1% of 10-Oxo-10,11-dihydro-5H-dibenzo [b,f]azepine-5-carboxamide impurity.

Zeta potential is a very important and highly desirable formulation technical attribute for a successful suspension composition development. Oral suspension compositions having zeta potential in between +30 or −30 mV are considered unstable, while oral suspension compositions having zeta potential outside +30 or −30 mV are considered stable.

The use of a suspending agent and its concentration has been found herein to affect the zeta-potential of the suspension composition. The present inventors surprisingly found that eslicarbazepine suspension compositions with only microcrystalline cellulose, carboxymethyl cellulose sodium, and a mixture thereof as suspending agent have stable pH, viscosity, and zeta-potential profile as compared to the suspension compositions having other suspending agents such as xanthan gum and carbomers. A clear phase separation was found for suspension compositions other than microcrystalline cellulose and carboxymethyl cellulose sodium as a suspending agent which clearly indicates failure in successful suspension formulation development with other suspending agents. The suspension compositions with only microcrystalline cellulose and carboxymethyl cellulose sodium as a suspending agent were free of 1) any phase separation, 2) any significant drop in pH, viscosity, and 3) free from impurities or having impurities within acceptable limits.

In some embodiments, the immediate release oral pharmaceutical ready to use suspension has a zeta potential value of +30 to +70 mV. In some embodiments, the immediate release oral pharmaceutical ready to use suspension has a zeta potential value of −30 to −70 mV. In some embodiments, the immediate release oral pharmaceutical ready to use suspension has a zeta potential value of +40 to +60 mV. In some embodiments, the immediate release oral pharmaceutical ready to use suspension has a zeta potential value of +50 to +60 mV. In some embodiments, the immediate release oral pharmaceutical ready to use suspension has a zeta potential value of +55 to +65 mV. Preferably, the immediate release oral pharmaceutical ready to use suspension has a zeta potential value of +57 mV. The zeta potential was determined by measuring the electrophoretic mobility of the particles using ZetaSizer Pro Blue, Malvern by taking about 1 ml of sample into a suitable size volumetric flask of about 250 mL and making up the volume with water. Collecting the sample solution by filtering the solution through a suitable glass fiber syringe filter (0.45 micron) after discarding the first 5 mL filtrate. The next step was filling the cuvette with the syringe as per the procedure mentioned in the instrument manual.

Liquid oral suspension compositions are susceptible to microbial contamination, which generally is controlled by adjustment of the pH of the preparation or adding one or more preservatives. However, the use of preservatives can degrade in suspension compositions depending on the pH. An alternative solution to this problem is to add higher amounts of preservatives. However, this may adversely affect the organoleptic properties i.e. taste of the suspension compositions, since preservatives generally have a bitter taste. This may result in lower patient compliance. Thus, there is an unmet need to develop an oral liquid suspension composition having both acceptable taste and low susceptibility to microbial contamination over the shelf life of the product.

The present inventors based on their extensive research have now discovered that the addition of a specific preservative up to a specific concentration to the eslicarbazepine acetate suspension compositions provides excellent preservative efficacy. The compositions possess sufficient antimicrobial activity to satisfy preservative efficacy requirements, as well as similar preservative standards.

As used herein, a suspension composition “having preservative efficacy is the one that is judged as “having preservative efficacy’ in the above-mentioned preservatives-effectiveness tests as specified in the USP, BP, EP, and Japanese pharmacopeia. In one embodiment, the preservative efficacy of the present preparations is maintained throughout their shelf-life. The immediate release oral ready to use suspension pharmaceutical compositions of eslicarbazepine acetate have the preservative efficacy which complies with ANTIMICROBIAL EFFECTIVENESS TESTING (preservatives-effectiveness tests) of “Microbiological tests' specified in the <51> United States Pharmacopeia (USP) 32.

In one embodiment, the enhancement of the preservative efficacy is evidenced by the reduction in viable cell count of Staphylococcus aureus (S. aureus), Escherichia coli, Pseudomonas aeruginosa, and fungi, such as Candida albicans and Aspergillus brasiliensis (niger).

In one embodiment, the composition has preservative efficacy, and the concentration of one or more preservatives is 25% or more. In one embodiment, the composition has preservative efficacy, and the concentration of one or more preservatives is from about 26% to 150%. In one embodiment, the composition does not provide sufficient preservative efficacy when the concentration of one or more preservatives is less than 30% or preferably less than 25%.

In one embodiment, there is provided an immediate release oral ready to use pharmaceutical suspension composition comprising:

a) eslicarbazepine acetate present at from about 0.1% to about 40% w/v;

b) a suspending agent present at from about 0.01% to about 10% w/v;

c) a surfactant present at from about 0.01% to about 7% w/v; and

d) a pharmaceutically acceptable liquid carrier present at from about 10% to about 95% w/v;

wherein pH of the suspension is from 3 to 6.5 and buffer concentration is from about 5 mM to about 25 mM and the suspension is free of suspending agents selected from the group consisting of xanthan gum and carbomer. In one embodiment, the viscosity of the suspension is from about 100 cps to 1000 cps. In one embodiment, the suspension has a zeta potential value of +50 to +65 mV. In one embodiment, the suspension has a sedimentation volume ratio of more than about 0.9 for at least 10 hours after the suspension is prepared. In one embodiment, the suspension has preservative efficacy and the concentration of one or more preservatives is 25% or more. In one embodiment, the preservative efficacy is evidenced by the reduction in viable cell count of Staphylococcus aureus (S. aureus), Escherichia coli, Pseudomonas aeruginosa, and fungi, such as Candida albicans and Aspergillus brasiliensis (niger). In one embodiment, the suspension comprises less than 1% of SLB-1 impurity.

In one embodiment, there is provided an immediate release oral ready to use pharmaceutical suspension composition comprising:

a) from about 0.1% to about 20% w/v of eslicarbazepine acetate;

b) from about 0.01% to about 10% w/v of one or more suspending agents selected from the group consisting of cellulose derivatives, co-processed spray-dried forms of microcrystalline cellulose and carboxymethyl cellulose sodium, gelatin, polyethylene glycols, polyvinyl compounds, sugar alcohols, colloidal silica, maltodextrin, starch, and combinations thereof;

c) from about 0.01% to about 7% w/v of one or more surfactants selected from the group consisting of sodium lauryl sulphate, polyethylene glycols, polyglycerin fatty acid, sorbitan fatty acid esters, polyoxyethylene sorbitan fatty acid esters, polyoxyethylene alkyl ethers, polyoxyethylene castor oil, polyoxyethylene-polyoxypropylene block copolymers, and combinations thereof;

d) from about 0.01% to about 15% w/v of one or more pH adjusting agents selected from the group consisting of citrate buffer, phosphate buffer, monosodium dibasic phosphate, citric acid, trisodium citrate, acetic acid, sodium phosphate, sodium citrate, sodium dihydrogen phosphate and potassium dihydrogen phosphate, hydrochloric acid, sodium hydroxide, and combinations thereof;

e) from about 0.001% to about 4% w/v of one or more preservatives selected from the group consisting of parabens and their salts, benzoic acid, sodium benzoate, potassium benzoate, methyl hydroxybenzoate, ethyl para-hydroxybenzoate, chlorhexidine, butylated hydroxyl toluene, butylated hydroxyl anisole, tocopherol, quaternary compounds, and combinations thereof;

f) from about 0.01% to about 70% w/v of one or more sweetening agents selected from the group consisting of thaumatin, sorbitol, and combinations thereof;

g) from about 0.01% to about 5% w/v of one or more flavoring agents; and

h) from about 10% to about 95% w/v of a pharmaceutically acceptable liquid carrier selected from the group consisting of water, glycerin, or a combination of water and glycerin;

wherein pH of the suspension is from 3 to 6.5, buffer concentration is from about 10 mM to about 20 mM, a zeta-potential value of +50 to +65 mV, and wherein suspension is free of xanthan gum, carbomer, and polyoxyethylene stearate.

In one embodiment, there is provided an immediate release oral ready to use pharmaceutical suspension composition free of xanthan gum, carbomer, and polyoxyethylene stearate comprising:

a) eslicarbazepine acetate present at from about 10 mg/ml to about 100 mg/ml;

b) one or more suspending agents selected from the group consisting of a mixture of microcrystalline cellulose and carboxymethyl cellulose sodium, propylene glycol alginate, starch, and combinations thereof present at from about 0.1 mg/ml to about 35 mg/ml;

c) one or more surfactants selected from the group consisting of sodium lauryl sulphate, sorbitan fatty acid esters, and polyoxyethylene sorbitan fatty acid esters, and combinations thereof present at from about 0.01 mg/ml to about 10 mg/ml;

d) one or more pH adjusting agents selected from the group consisting of citric acid, trisodium citrate, sodium citrate, hydrochloric acid, sodium hydroxide, and combinations thereof present at from about 0.01 mg/ml to about 10 mg/ml;

e) one or more preservatives selected from the group consisting of parabens and their salts, benzoic acid, sodium benzoate, and combinations thereof present at from about 0.001 mg/ml to about 10 mg/ml;

f) one or more sweetening agents selected from the group consisting of thaumatin, sorbitol, and a combination thereof present at from about 10 mg/ml to about 300 mg/ml;

g) one or more flavoring agents present at from about 0.001 mg/ml to about 10 mg/ml; and

h) a combination of water and glycerin as a pharmaceutically acceptable liquid carrier, wherein pH of the suspension is from 3 to 6.5, buffer concentration is from about 5 mM to about 25 mM, and suspension composition has a zeta potential value of +50 to +65 mV.

In one embodiment, there is provided an immediate release oral ready to use pharmaceutical suspension, comprises:

a) about 100 mg/ml of eslicarbazepine acetate;

b) about 12.5 mg/ml of a mixture of microcrystalline cellulose and carboxymethyl cellulose sodium;

c) about 0.4 mg/ml of polysorbate 80;

d) about 2.4 mg/ml of citric acid and trisodium citrate;

e) about 2.5 mg/ml of methylparaben and propylparaben;

f) about 102 mg/ml of thaumatin and sorbitol;

g) about 3.2 mg/ml of one or more flavoring agents; and

h) a combination of water and glycerin as a pharmaceutically acceptable liquid carrier.

The present inventors have performed extensive research with the aim of conceiving an immediate release oral ready to use suspension comprising eslicarbazepine, that shall be as effective as the known compositions in its preventive or curative effect, then the known compositions. The advantages of the present invention include the ability to provide higher levels of drug absorption, and consistent dose-to-effect times, making the present formulation a significant improvement for the treatment of epilepsy, partial-onset seizures, and other uses as described herein.

In accordance with another embodiment of the present invention, there is provided an immediate release oral ready to use suspension comprising eslicarbazepine acetate that provides high bioavailability, low variability in Tₘₐₓ, low variability in Cₘₐₓ, and low variability in AUC.

In accordance with another embodiment of the present invention, there is provided an immediate release oral ready to use suspension comprising eslicarbazepine acetate, wherein the suspension is bioequivalent and/or therapeutically equivalent to the marketed eslicarbazepine acetate oral tablets 200 mg, 400 mg, 600 mg and 800 mg (APTIOM® tablet). In another embodiment, the present invention provides immediate release oral pharmaceutical ready to use suspension, which is bioequivalent to eslicarbazepine acetate oral tablets 200 mg, 400 mg, 600 mg, and 800 mg (APTIOM® tablet) in a bioavailability study in humans. In another embodiment, the present invention provides immediate release oral pharmaceutical ready to use suspension, wherein the bioavailability study is conducted in humans under fasted conditions. In another embodiment, the present invention provides immediate release oral pharmaceutical ready to use suspension, wherein the bioavailability study is conducted in humans under fed conditions.

The pharmacokinetic parameters are established by observing: a) a 90% Confidence Interval between 80% and 125% for the ratio (of Test Product vs. Reference Product) of the geometric mean of AUC₀₋ₜ, b) a 90% Confidence Interval between 80% and 125% for the ratio (of Test Product vs. Reference Product) of the geometric mean AUC_{0-inf}, c) a 90% Confidence Interval between 80% and 125% for the ratio (of Test Product vs. Reference Product) of the geometric mean Cₘₐₓ, and d) a combination of any of a) to c).

In one embodiment, the compositions show comparative pharmacokinetics exposure in terms of point estimates which fall between 80%-125%. In certain embodiments, each dosage form is bioequivalent and/or therapeutically equivalent to about 100 mg to 1600 mg dose of eslicarbazepine acetate administered by oral administration as immediate-release tablets.

In another embodiment, the present invention provides for an immediate release oral pharmaceutical ready to use suspension composition comprising eslicarbazepine acetate 100 mg/ml which is bioequivalent to eslicarbazepine acetate tablets 800 mg marketed under the trade name of APTIOM® (eslicarbazepine acetate oral tablets 200 mg, 400 mg, 600 mg, and 800 mg).

In one embodiment, the immediate release oral ready to use suspension pharmaceutical compositions when administered to a human is bioequivalent within the 80-125% confidence interval and with a statistical power of at least 80% to an 800 mg tablet of eslicarbazepine in a bioavailability study in humans.

In one embodiment, the immediate release oral ready to use suspension pharmaceutical compositions comprises eslicarbazepine acetate in an amount of about 0.1 mg/mL to about 400 mg/mL, and one or more pharmaceutically acceptable excipients, and wherein said composition, when administered to a human subject, provides a value of AUC₀₋ₜ test to reference least squares mean ratio where the 90% confidence interval is between 80% and 125% of the natural-log transformed AUC₀₋ₜ value obtained with said commercially available eslicarbazepine tablets, and a value of AUC_{0-inf} test to reference least squares mean ratio where the 90% confidence interval is between 80% and 125% of the natural-log transformed AUC_{0-inf} value obtained with said commercially available eslicarbazepine tablets.

In one embodiment, when a suspension according to an embodiment of the present invention is orally administered to humans, a mean Cₘₐₓ is in a range of 13,000 to 30,000 ng/mL. In another embodiment, when a suspension according to an embodiment of the present invention is orally administered to humans, a mean Cₘₐₓ is in a range of 13,000 to 20,000 ng/mL. In one embodiment, the composition exhibits a Cₘₐₓ of about 13927 ng/mL to about 19371 ng/mL, following administration of the suspension composition to an adult human under fasted conditions. In one embodiment, the composition exhibits a Cₘₐₓ of from about 13927 ng/mL to about 19371 ng/mL, following administration of the suspension composition to an adult human under fasted conditions in single-dose testing. In one embodiment, the composition exhibits a Cₘₐₓ of about 16649.4±2722.1 ng/mL.

In one embodiment, the composition exhibits an AUC₀₋ₜ of from about 2,60,000 to about 3,74,000 ng·hr/mL, following administration of the suspension composition to an adult human under fasted conditions. In one embodiment, the composition exhibits an AUC₀₋ₜ of from about 263004.1 ng·hr/mL to about 373252.5 ng·hr/mL, following administration of the suspension composition to an adult human under fasted conditions in single-dose testing. In one embodiment, the composition exhibits an AUC₀₋ₜ of about 318128.3±55124.2 ng·hr/mL.

In one embodiment, the composition exhibits an AUC_{0-inf} of from about 200000 ng·hr/mL to about 400000 ng·hr/mL, following administration of the suspension composition to an adult human under fasted conditions. In one embodiment, the composition exhibits an AUC_{0-inf} of from about 276649.4 ng·hr/mL to about 399257.8 ng·hr/mL, following administration of the suspension composition to an adult human under fasted conditions in single-dose testing. In one embodiment, the composition exhibits an AUC_{0-inf} of about 337953.6±61304.2 ng·hr/mL.

In certain embodiments, the subject or patient is a human. In certain embodiments, the subject or patient is an adult. In certain embodiments, the subject or patient is an adolescent. In certain embodiments, the subject or patient is a child. In one embodiment, the subject or patient is of age group between one-year-old to 90 years old. In one embodiment, the compositions of the present invention can be given to diabetic patients because the composition is free of sugar.

In certain embodiments, the taste of oral suspension formulation of eslicarbazepine is acceptable when measured by the electronic tongue (e-tongue) method. The taste of oral suspension formulations of eslicarbazepine has been summarized as being bitter with a gritty mouth feel. The taste perception of the suspension formulation can be assessed by the electronic tongue (e-tongue) such as Astree® and/or Insent taste sensing system or any other known method known to skilled in the art. The e-tongue measures the relative repartition and proximity of the taste between an active suspension formulation and its matching placebo. The e-tongue measurements are usually analyzed by principal component analysis (PCA). Since the bitter active component is not present, it is assumed that the placebo represents the ideal “target” taste profile. Therefore, masking of bitterness or taste proximity is quantified using the Euclidean distance between the active and placebo formulations on the PCA map, with a smaller distance indicating a flavor that is doing a better job of masking and therefore bringing the active and placebo e-Tongue “fingerprint” closer together. The discrimination index (DI in %) takes into account the difference between the center of gravity of the sensors output for each pair of formulations as well as the dispersion within the sensors output for the formulations. The higher the value of discrimination index (closer to 100%), the less similarity between the formulations and less masking occurred. The electronic tongue uses seven sensor probe assemblies to detect dissolved compounds. These probes consist of silicon transistors with an organic coating that governs the sensitivity and selectivity of the probe. Measurements are made by potentiometric titration using measurements recorded against an Ag/AgCl reference electrode. Samples are placed in a rotating tray of an autosampler where electrodes are introduced into each sample. As applied to all taste features, each probe has cross-selectivity. Taste perception does not occur with the probe, but with the computer, where the electronic tongue statistics software translates the sensor data into taste patterns. Multiple different flavors and combinations are tested in the suspension formulation and its matching placebo to assess their masking efficiency. Not all the flavors can decrease the distance of the unmasked eslicarbazepine suspension. The discrimination indices show that the specific mixture of thaumatin (sweetener), sorbitol (sweetener), and flavoring agents as per the present invention are the most effective in masking the bitter taste of eslicarbazepine suspension. These results are in concordance with a human taste panel.

Carrier/vehicle/solvent used in the suspension of the present invention include aqueous and non-aqueous carriers but are not limited to water, alcohol, polyethylene glycol, propylene glycol or glycerin, buffers, oil, or combinations thereof. Oils include peanut oil, soy bean oil, corn oil, sesame oil, cottonseed oil, acetylated glycerides, ethyl oleate, mineral oil, fatty acid esters, mono- or di-fatty acid esters of polyethylene glycols, or glyceryl mono-oleate. Particularly, the suspensions are aqueous based. By “aqueous carrier” is meant a suspension comprising water, or a combination of water and a water-miscible organic solvent or solvents. Water-miscible solvents include but are not limited to propylene glycol, polyethylene glycol, and ethanol. By “non-aqueous carrier” is meant for a suspension in which the carrier does not include water. The carrier can also include one more pharmaceutically acceptable excipients which can be in dissolved or dispersed form. In particular, the carrier is water or a combination of water and glycerin. The carrier is present in an amount from about 5% w/w to about 99% w/w, about 10% w/w to about 99% w/w, particularly from about 30% w/w to about 95% w/w, particularly from about 40% w/w to about 95% w/w, particularly from about 50% w/w to about 95% w/w and particularly from about 60% w/w to about 95% w/w.

The viscosity agent/suspending agent enhances the physical stability of the composition by sufficiently increasing the viscosity to retard the settling rate, yet allowing adequate pourability. They also allow the product to be easily resuspendable so that an appropriate dose can be delivered with minimal shaking. Suitable thickening agents/viscosity agents/suspending agents are selected from the group comprising cellulose derivatives such as hydroxyethyl cellulose, hydroxypropyl cellulose, hydroxypropylmethyl cellulose, methylcellulose, carboxymethyl cellulose and its salts/derivatives e.g., carboxymethyl cellulose sodium, microcrystalline cellulose, and co-processed spray-dried forms of microcrystalline cellulose and carboxymethyl cellulose sodium (such as AVICEL® RC-501, AVICEL® RC-581, AVICEL® RC-591, and AVICEL® CL-611); carbomers (such as those available under the trade name CARBOPOL®); gums such as locust bean gum, tragacanth gum, arabinogalactan gum, agar gum, gellan gum, guar gum, apricot gum, karaya gum, sterculia gum, acacia gum, gum Arabic, and carrageenan; pectin; propylene glycol alginate, dextran; gelatin; polyethylene glycols; polyvinyl compounds such as polyvinyl acetate, polyvinyl alcohol, and polyvinyl pyrrolidone; sugar alcohols such as xylitol and mannitol; colloidal silica; maltodextrin, starch; and mixtures thereof. The liquid compositions of the present invention are free of xanthan gum. In a preferred embodiment, the suspending agent is a co-processed spray-dried form of microcrystalline cellulose and carboxymethyl cellulose sodium. The suspending agents/viscosity agents are present in an amount of about 0.01% to about 20% w/w of the composition. Particularly, the viscosity agents are present in an amount of about 0.01% to about 10% w/w of the composition. More particularly, the viscosity agents are present in an amount of about 0.1% to about 5% w/w of the composition. Much more particularly, the viscosity agents are present in an amount of about 0.1% to about 3% w/w of the composition.

The suspension is easily pourable and when shaken has a viscosity in the range of 100 to 5000 cps at 25° C. Particularly, the viscosity is in the range of 100 to 2500 cps at 25° C. Particularly, the viscosity is in the range of 100 to 1500 cPs at 25° C. More particularly, the viscosity is in the range of 700-1200 cps at 25° C.

The term “shaken” as used herein refers to shaken prior to use, e.g. by a patient or pharmacist, e.g. vigorously shaken, e.g. by hand, e.g. for at least 5 to 40 seconds.

The viscosity can be measured by using a suitable instrument such as Brookfield viscometer and Haake VT 550 viscometer at room temperature (25° C.).

Diluents or fillers are substances which usually provide bulk to the composition. Various useful fillers or diluents include, but are not limited to sucrose, sugar alcohols, sorbitol, xylitol, erythritol, starch, pregelatinized starch, calcium carbonate, calcium phosphate, dibasic anhydrous, calcium phosphate, dibasic dihydrate, calcium phosphate tribasic, calcium sulphate, cellulose powdered, silicified microcrystalline cellulose, cellulose acetate, lactose, magnesium carbonate, magnesium oxide, maltodextrin, mannitol, microcrystalline cellulose, silicified microcrystalline cellulose polydextrose, sodium alginate, sodium chloride and or mixtures thereof. Preferably the diluent used is sucrose or a sugar alcohol. The diluent is present in an amount of 5 to 98% w/w of the total composition.

The amount of surfactant or wetting agent should be sufficient to facilitate the dispersion of eslicarbazepine in the suspension. At the same time, it should provide improved wettability of the eslicarbazepine acetate. Suitable surfactant or wetting agents are selected from the group comprising non-ionic, anionic, cationic, or zwitterionic surfactants, and combinations thereof. Suitable examples of wetting agents are sodium lauryl sulphate; cetrimide; polyethylene glycols; polyglycerin fatty acid esters such as decaglyceryl monolaurate and decaglyceryl monomyristate; sorbitan fatty acid esters such as sorbitan monostearate; polyoxyethylene sorbitan fatty acid esters such as polyoxyethylene sorbitan monooleate; polyoxyethylene alkyl ether such as polyoxyethylene lauryl ether; polyoxyethylene castor oil; polyoxyethylene-polyoxypropylene block copolymers such as poloxamers (e.g. Poloxamer 188); and combinations thereof. Particularly, surfactants or wetting agents are non-ionic. The liquid compositions of the present invention are free of polyoxyethylene stearate such as polyoxy 100 stearate (MYRJ® 59P) as the wetting agent. The surfactant or wetting agents are present in an amount of about 0.01% to about 7% w/w of the composition. Particularly, the surfactant or wetting agents are present in an amount of about 0.01% to about 3% w/w, and more particularly from about 0.01% to about 1% w/w of the composition.

Various useful preservatives include, but are not limited to, parabens such as methylparaben, propylparaben, butyl paraben, and their salts, sorbic acid, sodium sorbate, potassium sorbate, calcium sorbate, benzoic acid, sodium benzoate, potassium benzoate, calcium benzoate, methyl hydroxybenzoate, ethyl para-hydroxybenzoate, sodium ethyl para-hydroxybenzoate, sodium metabisulphite, chlorhexidine, diazolidinyl urea, sodium citrate, butylated hydroxyl toluene (BHT), butylated hydroxyl anisole (BHA), tocopherol, ethylenediamine tetraacetic acid, propyl gallate, quaternary compounds, e.g. benzalkonium chloride and cetylpyridinium chloride, phenyl ethyl alcohol and combinations thereof. In particular, the preservative is selected from benzoic acid and its salts and parabens. The preservative is present in an amount of about 0.001% w/w to about 4% w/w of the composition, particularly in an amount of about 0.001% w/w to about 3% w/w of the composition.

Anticaking agent helps to improve the re-suspendability of the formulation. Various useful Anticaking agents include, but are not limited to, colloidal silica and/or colloidal silicon dioxide, calcium phosphate tribasic, magnesium oxide, magnesium silicate, calcium silicate, and combinations thereof. The anticaking agents are present in an amount of about 0.1% to about 10% w/w of the composition. More particularly, the anticaking agents are present in an amount of about 0.5% to about 7% w/w of the composition.

Various useful antioxidants include, but are not limited to, ascorbic acid, tert-butylhydroquinone, sodium pyrosulfite, glutathione, sodium bisulfite, sodium sulfite, a-tocopherol, a-tocopherol acetate, monothioglycerol, cysteine, ascorbyl palmitate, acetylcysteine, dithiothreitol, sodium metabisulfite, thiourea, sodium thiosulfate, butylated hydroxy anisole (BHA), butylated hydroxytoluene (BHT) and propyl gallate. The antioxidants are present in an amount of 0% to about 10% w/w of the composition.

Various useful sweetening agents include, but are not limited to, sugars such as sucrose, dextrose, fructose, lactose, maltose, invert sugar; sugar alcohols such as sorbitol, mannitol, xylitol, lactitol, erythritol, maltodextrin, maltitol, isomaltitol, isomalt, maltulose, isomaltulose, lactulose, threitol, arabitol, ribitol, galactitol, and sugar substitutes such as saccharin sodium, aspartame, thaumatin, acesulfame, combinations thereof. Sugar or sugar alcohol can also act as a filler. Preferably sweetening agent used is sodium saccharin. The sweetening agents are present in an amount of about 0.01% w/w to about 99% w/w of the composition, particularly in an amount of about 0.1% w/w to about 90% w/w of the composition, particularly in an amount of about 0.01% w/w to about 80% w/w of the composition, particularly in an amount of about 0.1% w/w to about 80% w/w of the composition, particularly in an amount of about 0.1% w/w to about 70% w/w of the composition, particularly in an amount of about 0.1% w/w to about 60% w/w of the composition, particularly in an amount of about 0.1% w/w to about 50% w/w of the composition, particularly in an amount of about 0.1% w/w to about 40% w/w of the composition and more particularly in an amount of about 0.1% w/w to about 30% w/w of the composition. Thaumatin or a combination of sorbitol and thaumatin as the sweetening agent is preferable.

Various useful flavoring agents, include, but are not limited to, flavors such as banana, lemon, orange, grape, lime and grapefruit, vanilla, bubble gum, peppermint, fantasy fruit masking flavor (as commercially available), and fruit essence, including apple, banana, pear, peach, strawberry, raspberry, cherry, plum, pineapple, apricot; synthetic flavor oils and flavoring aromatics and/or natural oils, extracts from plant leaves, flowers, fruits such as cinnamon oil, oil of wintergreen, peppermint oils, clove oil, citrus oil, bay oil, anise oil, eucalyptus, thyme oil, cedar leaf oil, oil of nutmeg, oil of sage, oil of bitter almonds, and cassia oil; maltol, ethyl vanillin, menthol, citric acid, fumaric acid, ethyl maltol, and tartaric acid and combinations thereof. The flavoring agents are present in an amount of about 0.01% to about 7% w/w of the composition. Particularly, the flavoring agents are present in an amount of about 0.01% to about 5% w/w of the composition, about 0.01% to about 3% w/w of the composition about 0.01% to about 2% w/w of the composition, and particularly from about 0.01% to about 1% w/w of the composition.

Various useful isotonicity adjusting agents include, but are not limited to, sodium chloride, mannitol, D-sorbitol, glucose, glycerin, or the like.

Various useful pH adjusting agents or buffering agents include, but are not limited to, citrate buffers, phosphate buffers, or any other suitable buffer known in the art including monosodium dibasic phosphate, gluconic acid, lactic acid, citric acid, trisodium citrate, acetic acid, maleic acid, tartaric acid, fumaric acid, sodium phosphate, sodium gluconate, sodium lactate, sodium citrate, sodium acetate potassium citrate, sodium bicarbonate, potassium bicarbonate, sodium dihydrogen phosphate and potassium dihydrogen phosphate, and combinations thereof. They also include a combination of acidic and basic substances. Strong acids such as hydrochloric acid, sulfuric acid, phosphoric acid, and the like can be used alone or in combination with basic substances such as inorganic bases (e.g., sodium hydroxide, potassium hydroxide, sodium carbonate, sodium hydrogen carbonate, magnesium carbonate, calcium carbonate, magnesium oxide, ammonia, synthetic hydrotalcite), organic bases (e.g., basic amino acids such as lysine, arginine, etc., meglumine, and the like).

The pH adjusting agent or buffering agents are present in an amount of about 0.001% to about 15% w/w of the composition, particularly from about 0.01% to about 10% w/w of the composition, from about 0.01% to about 8% w/w of the composition, from about 0.01% to about 5% w/w of the composition, particularly from about 0.01% to about 3% w/w of the composition and particularly from about 0.005% to about 3% w/w of the composition. In particular, citrate and trisodium citrate buffer is used. The pH of the eslicarbazepine suspension ranges from about 2 to about 8. Preferably, the pH of the eslicarbazepine suspension ranges from 3.0 to 6.5. In an embodiment, the buffer concentration is in the range of about 0.5 mM to about 40 mM. Particularly, the buffer concentration is in the range of about 2 mM to about 20 mM.

Various useful taste masking agents include, but are not limited to, water soluble and/or insoluble polymeric excipient, water insoluble non-polymeric excipient, adsorbent, ion exchange resins such as AMBERLITE® (ion exchange resins), AMBERLITE® CG 50 (weakly acidic, carboxylic acid type of cation exchange resin), AMBERLITE® IRP-64 (cationic ion exchange resin that is derived from a copolymer of methacrylic acid and divinyl benzene), AMBERLITE® IRP-69 (sodium polystyrene sulfonate or sulfonated copolymer of styrene and divinylbenzene), INDION® 204 (crosslinked polyacrylic acid), INDION® 214 (crosslinked polyacrylic acid), INDION® 234 (crosslinked polyacrylic acid), INDION® CRP 244 (Polystyrene matrix cation-exchange resins), INDION® CRP 254 (Polystyrene matrix cation-exchange resins), carbomers such as Carbomer 934, Carbomer 974, Carbomer 971, PEG-5M, CARBOPOL®934P NF (Carbomer 934P, NF), CARBOPOL®971P (Carbomer 971P, NF), CARBOPOL® 974P NF (Carbomer 974P, NF), alkali metal chlorides or an alkaline earth metal chloride or a derivative thereof, OPADRY® AMB (polyvinyl alcohol or copolymers or mixtures thereof—a water soluble coating material available from Colorcon, Pa., USA), sodium starch glycolate, bitter masker US 151 flavor, PEG-5M, sodium acetate, ethylcellulose, cyclodextrin, beta-cyclodextrin, polyvinyl acetate dispersion, trehalose, vinylacetate, polystyrene, cellulose acetate butyrate, methacrylic acid, and methyl methacrylates such as EUDRAGIT® R) L100, polymethacrylates (such as EUDRAGIT® (R) L100), sodium chloride, polyethoxylated castor oil, KOLLIPHOR® RH 40 (Polyoxyl 40 hydrogenated castor oil), SENTRY® POLYOX® WSR N80 NF (poly (ethylene oxide)), natural or synthetic fatty type or other flavorant such as cocoa, chocolate (especially mint chocolate), cocoa butter, milk fractions, vanillin butterfat, egg or egg white, peppermint oil, wintergreen oil, spearmint oil, and similar oils. Compounds which reduce throat catch include for example high solubility acids include amino acids (e.g. alanine, arginine, etc.), glutaric, ascorbic, malic, oxalic, tartaric, malonic, acetic, citric acids, low solubility acids include oleic, stearic, and aspartic acids plus certain amino acids such as glutamic acid, glutamine, histidine, isoleucine, leucine, methionine, phenylalanine, serine, tryptophan, tyrosine, valine, and fumaric acid. The taste-masking agents are present in an amount of about 0.01% to about 50% w/w of the composition, particularly from about 0.01% to about 30% w/w of the composition, particularly from about 0.01% to about 20% w/w of the composition, and particularly from about 0.01% to about 10% w/w of the composition.

Various useful antifoaming agents include, but are not limited to simethicone.

The pharmaceutical composition of the present invention can be packaged in a suitable pack/container such as amber colored polyethylene terephthalate (PET) bottle, glass bottle, high density polyethylene (HDPE) bottle, low density polyethylene (LDPE) bottle, polypropylene (PP) bottle, packets, pouches, sachets and the like. The glass or plastic bottle is provided with a child proof closure. The package can include a syringe, dosing syringe or dispensing syringe or measuring cup, or any combination (marked in mL) for ease of dosing. The container(s) of the present invention may have a syringe or cup adapted to be attached to the container. The syringe or cup as per the present invention can be of material such as polyethylene terephthalate (PET), glass, high density polyethylene (HDPE), low density polyethylene (LDPE), polypropylene (PP), or any other material known in the art.

The container such as bottle has a fill volume of, e.g., from about 50 mL to about 500 mL comprising eslicarbazepine suspension. Packs chosen are made of material which is non-reactive with the suspension and suspension powder for reconstitution. Containers for use in the storage of the oral suspensions may be used to administer a multiple dose of eslicarbazepine.

The liquid pharmaceutical composition of the present invention can be used for the treatment of seizures, partial-onset seizures, epilepsy, neuropathic pain, migraine, fibromyalgia, trigeminal neuralgia, bipolar disorders, attention disorders, anxiety disorders, affective disorders, schizoaffective disorders, sensorimotor disorders, and vestibular disorders.

The compositions of the present invention are for oral administration. The compositions may be taken in measured doses using a container, cup, straw, spoon, syringe, dispensing syringe, dosing syringe, or any other suitable device. The compositions may be provided in liquid form, or in dry form (such as granule or powder or multiparticulate) to which water or liquid solvent or diluent is added to provide a liquid composition of this invention. Ingredients suitable for liquid compositions are known and such compositions may be made by methods known in the art. In an embodiment, the syringe, dispensing syringe, or dosing syringe, or combination thereof are used to transfer a predetermined amount of the composition comprising eslicarbazepine or its salt thereof, into the patient's mouth. In an embodiment, the measuring cup is used to measure the dose as per the patient's requirement so that a precise dosage can be obtained for oral administration.

Suitable coloring agents are selected from the group comprising FD&C (Federal Food, Drug and Cosmetic Act) approved coloring agents; natural coloring agents; natural juice concentrates; pigments such as iron oxide, titanium dioxide, and zinc oxide; and combinations thereof.

The suspensions of the present invention are homogenous and deliver the desired dose of eslicarbazepine in every use without any risk of overdosing or underdosing. The compositions provide predictable eslicarbazepine release throughout the shelf life.

The invention may be further illustrated by the following examples, which are for illustrative purposes only and should not be construed as limiting the scope of the invention in any way.

### EXAMPLES

### Example 1—Ready to Use Suspension

| Ingredients | Quantity (% w/w) |
|---|---|
| Eslicarbazepine acetate | 6.70 |
| Sucrose | 26.80 |
| Sorbitol | 40.20 |
| Poloxamer 188 | 1.10 |
| Propylene glycol | 15.01 |
| Polysorbate 80 | 4.70 |
| Citric acid | 0.06 |
| Monosodium dibasic phosphate | 0.03 |
| Sodium saccharin | 0.08 |
| Methyl paraben | 0.16 |
| Propyl paraben | 0.11 |
| AVICEL ® RC591 (Microcrystalline | 0.89 |
| cellulose and sodium | |
| carboxymethylcellulose)/Sodium | |
| carboxymethylcellulose | |
| Acesulfame potassium | 1.61 |
| Bubble gum flavor | 0.04 |
| Purified water | q.s. |

Procedure:

1. Polysorbate 80 and eslicarbazepine acetate were mixed.

2. Propylene glycol and Poloxamer 188 were added to step 1 and mixed.

3. Sodium saccharin, mono sodium dibasic phosphate anhydrous, and citric acid were added to purified water to form a solution.

4. Methyl paraben and propyl paraben were added to propylene glycol to obtain a clear solution.

5. AVICEL® RC 591 (Microcrystalline cellulose and sodium carboxymethylcellulose)/sodium carboxymethylcellulose was added to water.

6. Solution of step 3 was added to step 2 and mixed.

7. Solution of step 4 was added to step 6 and mixed.

8. Dispersion of step 5 was added to step 7 and mixed.

9. Sucrose, sorbitol, acesulfame potassium, and bubble gum flavor were added to water and mixed with the mixture of step 8 and homogenized.

### Examples 2-6—Suspension Powder for Reconstitution

| | Quantity (% w/w) | | | | |
|---|---|---|---|---|---|
| Example | 2 | 3 | 4 | 5 | 6 |
| Eslicarbazepine acetate | 12.50 | 12.50 | 12.50 | 12.50 | 12.50 |
| Sucrose | 86.30 | 79.60 | 79.53 | 78.72 | 78.48 |
| Polysorbate 80 | — | — | 0.08 | — | — |
| Citric acid | 0.11 | 0.11 | 0.11 | 0.11 | — |
| Monosodium dibasic | 0.06 | 0.06 | 0.06 | 0.06 | — |
| phosphate | | | | | |
| Tribasic sodium phosphate | — | — | — | — | 0.11 |
| Sodium saccharin | — | 0.15 | 0.15 | 0.15 | 0.15 |
| Sodium benzoate | — | 0.30 | 0.30 | 0.30 | 0.30 |
| AVICEL ® RC591 | 0.88 | 0.88 | 0.88 | — | 1.75 |
| (Microcrystalline | | | | | |
| cellulose and sodium | | | | | |
| carboxymethylcellulose) | | | | | |
| Silicon dioxide | — | 6.25 | 6.25 | 6.25 | 6.25 |
| Propylene glycol alginate | — | — | — | 1.75 | — |
| Gellan gum | — | — | — | — | 0.3 |
| Banana flavor/Cherry flavor | 0.16 | 0.16 | 0.16 | 0.16 | 0.16 |

Procedure:

1. Eslicarbazepine acetate and sucrose were mixed.

2. Tribasic sodium phosphate/sodium saccharin/sodium benzoate/Polysorbate 80 were dissolved in water.

3. The mixture of step 1 was granulated with the solution of step 2.

4. The wet mass of step 3 was dried and milled to form granules.

5. Remaining excipients were added to the granules of step 4 and mixed.

The suspension powder for reconstitution is reconstituted with a suitable carrier when required.

### Examples 7-10—Ready to Use Suspension of Eslicarbazepine Acetate Procedure

| | | Quantity (% w/w) | | | |
|---|---|---|---|---|---|
| | | Example | Example | Example | Example |
| Ingredients | Function | 7 | 8 | 9 | 10 |
| Eslicarbazepine | Active | 0.5-40 | 10 | 5 | 10 |
| acetate | | | | | |
| Polysorbate 80 | Surfactant | 0.01-7 | 0.04 | 0.06 | 0.05 |
| AVICEL ® RC591 | Suspending | 0.01-10 | 1.25 | 2.75 | 2.25 |
| (Microcrystalline | agent | | | | |
| cellulose and | | | | | |
| sodium carboxy- | | | | | |
| methylcellulose) | | | | | |
| Sorbitol | Sweetener | 1-70 | 10 | 15 | 20 |
| Thaumatin | Sweetener | 0.01-2 | 0.2 | 0.09 | 0.15 |
| Methyl paraben | Pre- | 0.01-3 | 0.25 | 0.25 | 0.40 |
| or propyl paraben | servative | | | | |
| or combination | | | | | |
| thereof | | | | | |
| Citric acid | Buffering | 0.01-5 | 0.10 | 0.07 | 0.05 |
| | agent | | | | |
| Trisodium citrate | Buffering | 0.01-5 | 0.15 | 0.08 | 0.05 |
| | agent | | | | |
| Lemon flavor or | Flavoring | 0.01-3 | 0.32 | 0.18 | 0.08 |
| vanilla flavor | agent | | | | |
| or peppermint | | | | | |
| flavor or fantasy | | | | | |
| fruit masking | | | | | |
| flavor or straw- | | | | | |
| berry flavor or | | | | | |
| bubble gum | | | | | |
| flavor or combi- | | | | | |
| nations thereof | | | | | |
| Glycerin | Carrier | 2-50 | 10 | 15 | 20 |
| Purified water | Carrier | q.s. | q.s | q.s | q.s |

Procedure:

1. Sorbitol, AVICEL® RC591, citric acid, and trisodium citrate were added to water with stirring to form a dispersion;

2. Methyl paraben and propyl paraben were added to glycerin with stirring;

3. Dispersion of step 2 was added to the dispersion of step 1 with stirring;

4. Eslicarbazepine acetate and polysorbate 80 were added to water with stirring and optionally homogenized;

5. Dispersion of step 4 was added to the dispersion of step 3; and

6. Thaumatin and flavoring agents were added to the dispersion of step 5 and the remaining amount of water is added to form the suspension.

Assay of Eslicarbazepine

The suspension powder for reconstitution of Examples 1, 2, and 6 were analyzed for drug content by HPLC method using C18 column (150×4.6 mm, 5 μm) using acetonitrile:water (50:50) at 215 nm. The results are provided in Table 1.

TABLE 1
Assay for Eslicarbazepine

| Composition | % Assay |
|---|---|
| Example 1 | 102.9 |
| Example 2 | 99.7 |
| Example 6 | 101.70 |

pH data: pH values were determined using potentiometry using USP <791> with the results provided in Table 2.

TABLE 2
pH Value

| Composition | pH |
|---|---|
| Example 1 | 5.40 |
| Example 2 | 5.20 |
| Example 3 | 4.83 |
| Example 4 | 5.43 |
| Example 5 | 4.72 |
| Example 6 | 7.80 |
| Example 8 | 4.77 |

Dissolution Studies

The powders for suspension of Examples 1-6 were evaluated for in-vitro eslicarbazepine release. The in-vitro dissolution was determined using a USP type II apparatus (paddle) at 100 rpm in 1000 mL of acetate buffer (pH 4.5) at 37±0.5° C. by the HPLC method. The results are reported in Table 3.

TABLE 3
Percentage (%) of the In-Vitro Eslicarbazepine Release in USP type II
apparatus at 100 rpm in 1000 mL of acetate buffer (pH 4.5)
Percentage of Eslicarbazepine Release

| Time | Example | Example | Example | Example | Example | Example |
|---|---|---|---|---|---|---|
| (minutes) | 1 | 2 | 3 | 4 | 5 | 6 |
| 15 | 102 | 72 | 107 | 110 | 103 | 96 |
| 30 | 101 | 84 | 109 | — | 104 | 98 |
| 45 | 100 | 89 | 109 | — | 104 | 99 |

Viscosity: The viscosity of the eslicarbazepine suspension of Example 8 was determined to be 321 cps using Brookfield viscometer using spindle LV-3 (Spindle number 63) at 50 rpm and 25° C.

Sedimentation Ratio

The sedimentation ratio was determined for Example 8 and the results are provided in Table 4. To measure the sedimentation ratio, the eslicarbazepine suspension of Example 8 was filled up to 100 mL in a 100 mL measuring cylinders and covered with a stopper. The initial height of the sediment was measured as HO. After a particular time period (such as 1 day), measure the height of sediment as Hu. Table 4 reports the sedimentation ratio of eslicarbazepine suspension after 6 days. There is no change in the sedimentation ratio for at least 6 days. The sedimentation ratios of 1 at days 1 and 6 indicate that the suspension is physically stable.

Sedimentation ratio=Hu/HO

TABLE 4
Sedimentation ratio

| | Sedimentation ratio | |
|---|---|---|
| Composition | 1 day | 6 days |
| Example 8 | 1 | 1 |

Dissolution Data

The eslicarbazepine suspension of Example 8 was evaluated for in-vitro eslicarbazepine release. The in-vitro dissolution was determined using a USP type II apparatus at 100 rpm in 1000 mL of acetate buffer (pH 4.5) at 37±0.5° C. by a validated HPLC method. The results are reported in Table 5.

TABLE 5
Percentage (%) of the In-Vitro Eslicarbazepine Release in USP type II
apparatus at 100 rpm in 1000 mL of acetate buffer (pH 4.5)
Percentage of Eslicarbazepine Release

| Time (minutes) | Example 8 |
|---|---|
| 5 | 96 |
| 10 | 97 |
| 15 | 98 |
| 30 | 98 |

X-Ray Diffraction Study

### Example 11—Ready to Use Suspension of Eslicarbazepine Acetate

| Ingredients | % w/w |
|---|---|
| Eslicarbazepine acetate | 10.00 |
| Polysorbate 80 | 0.04 |
| Microcrystalline cellulose and | 2.00 |
| sodium carboxymethylcellulose | |
| Sorbitol | 10.00 |
| Thaumatin | 0.15 |
| Methyl paraben/propyl paraben/ | 0.25 |
| combination thereof | |
| Citric acid | 0.10 |
| Trisodium citrate | 0.15 |
| Lemon flavor or peppermint | 0.09 |
| flavor or fantasy fruit masking | |
| flavor or combinations thereof | |
| Glycerin | 10.00 |
| Purified water | q.s |

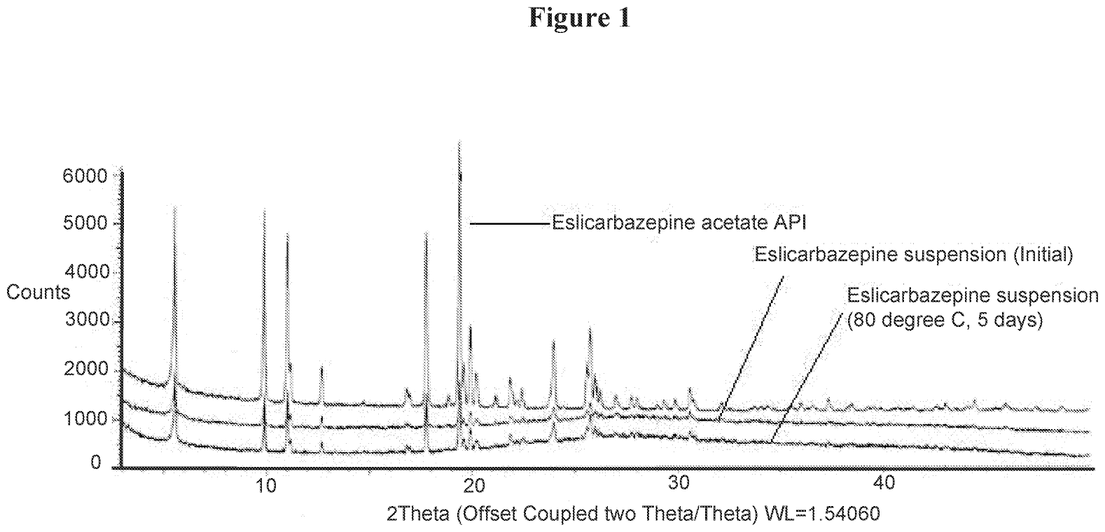

Bruker D8 Advance X-Ray diffractometer with a CuKα radiation was used for X-Ray diffraction study of eslicarbazepine acetate suspension. FIG. 1 shows an overlay of eslicarbazepine acetate drug substance and eslicarbazepine suspension (initial) and eslicarbazepine suspension stability samples (stored at 80° C. for 5 days) as per Example 11.

The peaks of eslicarbazepine acetate API was observed in eslicarbazepine suspension samples (initial and stability sample stored at 80° C. for 5 days) at 5.58°, 9.82°, 11.07°, 12.63°, 17.80°, 19.33°, and 21.91±0.2° 2Θ. Eslicarbazepine acetate was found to be stable in the suspension as no change in the XRD pattern of eslicarbazepine acetate was observed in the formulation.

Stability Studies

1. Stability Study of Eslicarbazepine Suspension of Example 8

Eslicarbazepine suspension prepared as per Example 8 was packed in a glass bottle and subjected to accelerated stability conditions 40° C./75% Relative Humidity (R.H.) and at 25° C./60% R.H. for 1 month. The results are provided in Table 6.

TABLE 6
Stability data of eslicarbazepine suspension of Example 8

| | | 40° C./75% R.H., | 25° C./60% R.H., |
|---|---|---|---|
| Test Parameter | Initial | 1 month | 1 month |
| pH | 4.77 | 4.69 | 4.67 |
| Viscosity | 321 | 370 | 351 |
| Related substances | 0.035 | 0.05 | 0.04 |
| (R.S.) | | | |
| Assay | 101.5 | 101.5 | 101.0 |

Dissolution (pH 4.5 acetate buffer, paddle, 1000 ml, 100 rpm)

| Time (minutes) | Percentage of Eslicarbazepine Release | | |
|---|---|---|---|
| 5 | 96 | 95 | 95 |
| 10 | 97 | 97 | 98 |
| 15 | 98 | 97 | 101 |
| 30 | 98 | 99 | 100 |

From the above data, it is clear that eslicarbazepine suspension is stable in a glass bottle when stored at 40° C./75% R.H. and 25° C./60% R.H. for a period of at least 1 month. Further, no phase separation was observed at these stability conditions.

2. Stability Study of Eslicarbazepine Suspension of Example 12

Eslicarbazepine suspension prepared as per Example 12 was packed in glass bottle and PET bottle and subjected to accelerated stability conditions 40° C./75% Relative Humidity (R.H.) for 1 month and 3 months and at 25° C./60% R.H. The results are provided in Table 7.

### Example 12—Ready to Use Suspension of Eslicarbazepine Acetate

| Ingredients | % w/w |
|---|---|
| Eslicarbazepine acetate | 5.00 |
| Polysorbate 80 | 0.04 |
| Microcrystalline cellulose and | 3.00 |
| sodium carboxymethylcellulose | |
| Sorbitol | 20.00 |
| Sucralose | 1.00 |
| Methyl paraben or propyl paraben | 0.25 |
| or combination thereof | |
| Citric acid | 0.02 |
| Trisodium citrate | 0.03 |
| Orange flavor | 0.30 |
| Glycerin | 10.00 |
| Purified water | q.s |

TABLE 7
Stability data eslicarbazepine suspension of Example 12

| | | Glass Bottle | | | PET Bottle | | |
|---|---|---|---|---|---|---|---|
| Pack | | 40° C./75% R.H. | | 25° C./60% R.H. | 40° C./75% R.H. | | 25° C./60% R.H. |
| Condition | Initial | 1 month | 3 months | 3 months | 1 month | 3 months | 3 months |
| Related | 0.05 | 0.14 | 0.27 | 0.06 | 0.13 | 0.25 | 0.06 |
| substances | | | | | | | |
| (R.S.) | | | | | | | |

Dissolution (pH 4.5 acetate buffer, paddle, 1000 ml, 100 rpm)

| Time | | | | | | | |
|---|---|---|---|---|---|---|---|
| (minutes) | Percentage of Eslicarbazepine Release | | | | | | |
| 5 | 99 | 99 | 99 | 98 | 98 | 96 | 100 |
| 10 | 99 | 99 | 99 | 98 | 98 | 96 | 100 |
| 15 | 100 | 99 | 99 | 98 | 98 | 96 | 100 |
| 30 | 99 | 99 | 99 | 98 | 98 | 96 | 100 |

The undesirable known Related substances (R.S.)/impurities in eslicarbazepine acetate comprise oxcarbazepine, carbamazepine, and dehydro eslicarbazepine and unknown impurities.

From the above data, it is clear that eslicarbazepine suspension is stable in glass bottle and PET bottle when stored at 40° C./75% R.H. and 25° C./60% R.H. for a period of at least 3 months. Further, no phase separation was observed at these stability conditions.

pH Stability Profile

pH stability profile of the ready to use suspension of eslicarbazepine acetate of Example 13 was studied. The data is represented in Table 8.

### Example 13—Ready to Use Suspension of Eslicarbazepine Acetate

| Ingredients | Quantity (% w/w) |
|---|---|
| Eslicarbazepine acetate | 5.00 |
| Polysorbate 80 | 0.04 |
| AVICEL ® RC591 | 2.75 |
| (Microcrystalline cellulose | |
| and sodium | |
| carboxymethylcellulose) | |
| Sorbitol | 20.00 |
| Methyl paraben or propyl | 0.25 |
| paraben or combinations | |
| thereof | |
| Citric acid | 0.04 |
| Trisodium citrate | 0.02 |
| Glycerin | 10.00 |
| Purified water | q.s. |

TABLE 8
pH stability profile of eslicarbazepine suspension

| Related | pH | pH | pH | pH | pH | pH | pH | pH | pH | pH |
|---|---|---|---|---|---|---|---|---|---|---|
| substances | 2.0 | 2.5 | 3.0 | 3.5 | 4.0 | 4.5 | 5.0 | 5.5 | 6.0 | 7.0 |
| Initial | 0.135 | 0.072 | 0.044 | 0.039 | 0.041 | 0.012 | 0.039 | 0.038 | 0.044 | 0.053 |
| 40° C./75% | 13.25 | 4.40 | 0.84 | 0.36 | 0.18 | 0.14 | 0.14 | 0.24 | 0.47 | 1.42 |
| R.H., 3 months | | | | | | | | | | |

Organoleptic Properties

Eslicarbazepine acetate is a bitter-tasting drug. To increase the patient acceptability of the ready to use suspension, the bitterness of the API needs to be masked by using a suitable combination of sweeteners and flavors. Organoleptic properties of the suspensions were evaluated for texture, taste, after taste, odor, flavor, and acceptability. The suspension of Example 13 comprises bubble gum flavor, peppermint flavor, fantasy fruit masking flavor, strawberry flavor, and thaumatin and was perceived to have a good overall acceptability on the basis of tested organoleptic properties.

The compositions disclosed herein can comprise, consist essentially of, or consist of, the recited or listed active ingredient and excipients.

### Examples (Ex.) 14-21—Ready to Use Suspension of Eslicarbazepine Acetate

| | | Quantity (% w/v) | | | | | | | |
|---|---|---|---|---|---|---|---|---|---|
| Ingredients | Function | Ex. 14 | Ex. 15 | Ex. 16 | Ex. 17 | Ex. 18 | Ex. 19 | Ex. 20 | Ex. 21 |
| Buffer Capacity | | 1.6 | 2.9 | 7.1 | 10.0 | 20.0 | 2.9 | 10.0 | 20.0 |
| Eslicarbazepine | Drug | 5 | 5 | 5 | 5 | 5 | 10 | 10 | 10 |
| Acetate | | | | | | | | | |
| Glycerine | Vehicle | 10 | 10 | 10 | 10 | 10 | 10 | 10 | 10 |
| Polysorbate 80 | Surfactant | 0.04 | 0.04 | 0.04 | 0.04 | 0.04 | 0.04 | 0.04 | 0.04 |
| Methyl paraben | Preservative | 0.2 | 0.2 | 0.2 | 0.2 | 0.2 | 0.2 | 0.2 | 0.2 |
| Propyl paraben | | 0.05 | 0.05 | 0.05 | 0.05 | 0.05 | 0.05 | 0.05 | 0.05 |
| Sorbitol | Sweetener | 20 | 10 | 20 | 10 | 10 | 10 | 10 | 10 |
| Sucralose | | 1 | — | 1 | — | — | — | — | — |
| Thaumatin | | — | 0.075 | — | 0.075 | 0.075 | 0.075 | 0.15 | 0.15 |
| Microcrystalline | Suspending | 3 | 2.75 | 2.75 | 2.75 | 2.75 | 2.0 | 2.25 | 2.25 |
| Cellulose and | agent | | | | | | | | |
| Carboxymethyl | | | | | | | | | |
| cellulose sodium | | | | | | | | | |
| Citric Acid | Buffering | 0.015 | 0.046 | 0.118 | 0.095 | 0.189 | 0.046 | 0.083 | 0.166 |
| Trisodium | agent | 0.025 | 0.014 | 0.03 | 0.15 | 0.299 | 0.014 | 0.167 | 0.335 |
| Citrate | | | | | | | | | |
| Flavoring Agents | Flavors | 0.25 | 0.038 | 0.15 | 0.048 | 0.048 | 0.038 | 0.086 | 0.136 |
| Purified water | Vehicle | q.s. | q.s. | q.s. | q.s. | q.s. | q.s. | q.s. | q.s. |

TABLE 9
Buffer concentration impact on eslicarbazepine suspension compositions

| | Example No. | | | | | | | | | | | | | | | |
|---|---|---|---|---|---|---|---|---|---|---|---|---|---|---|---|---|
| | Ex. 14 | | Ex. 15 | | Ex. 16 | | Ex. 17 | | Ex. 18 | | Ex. 19 | | Ex. 20 | | Ex. 21 | |
| | Buffer capacity (mM) | | | | | | | | | | | | | | | |
| | 1.6 | | 2.9 | | 7.1 | | 10 | | 20 | | 2.9 | | 10 | | 20 | |
| | Condition | | | | | | | | | | | | | | | |
| | | 80° C./ | | 80° C./ | | 80° C./ | | 80° C./ | | 80° C./ | | 80° C./ | | 80° C./ | | 80° C./ |
| | Initial | 5 days | Initial | 5 days | Initial | 5 days | Initial | 5 days | Initial | 5 days | Initial | 5 days | Initial | 5 days | Initial | 5 days |
| pH | 5.48 | 5.10 | 4.68 | 4.60 | 4.07 | 4.03 | 4.88 | 4.78 | 4.76 | 4.72 | 4.65 | 4.42 | 5.08 | 4.99 | 4.92 | 4.84 |
| Visco- | 865 | — | 520 | — | 142 | — | 356 | 307 | 259 | 213 | 930 | 404 | 830 | 617 | 682 | 534 |
| sity | | | | | | | | | | | | | | | | |
| Total | 0.052 | 1.566 | 0.043 | 0.829 | 0.047 | 1.048 | 0.044 | 0.665 | 0.042 | 0.598 | 0.039 | 0.608 | 0.041 | 0.139 | 0.036 | 0.24 |
| Im- | | | | | | | | | | | | | | | | |
| purities | | | | | | | | | | | | | | | | |

Results: As per the Table No. 9, it was observed that the composition containing higher buffer concentration had shown a stable pH profile, viscosity, and less degradation as compared to the composition containing less buffer concentration when measured at about 80° C. for at least 5 days.

### Example (Ex.) 22-24—Ready to Use Suspension of Eslicarbazepine Acetate

| | | Ex. 22 | Ex. 23 | Ex. 24 |
|---|---|---|---|---|
| Ingredients | Function | Quantity (% w/v) | | |
| Eslicarbazepine | Drug | 5 | 5 | 10 |
| Xanthan gum | Suspending | 0.3 | — | — |
| Microcrystalline | agent | — | — | 1.25 |
| cellulose and sodium | | | | |
| carboxymethyl | | | | |
| cellulose | | | | |
| Carbomer | | — | 0.2 | — |
| Sorbitol | Sweetener | 20 | 20 | 10 |
| Glycerin | Vehicle | 10 | — | 10 |
| Polysorbate 80 | Surfactant | 0.04 | 0.04 | 0.04 |
| Methyl paraben | Preservative | 0.2 | — | 0.2 |
| Propyl Paraben | | 0.05 | — | 0.05 |
| Sodium Benzoate | | — | 0.25 | — |
| Sucralose | Sweetener | 1 | 1 | — |
| Thaumatin | | — | — | 0.2 |
| Flavoring Agents | Flavors | 0.25 | 0.15 | 0.32 |
| Citric Acid | Buffering | 0.015 | — | 0.095 |
| Trisodium citrate | agent | 0.025 | — | 0.15 |
| Sodium Hydroxide | | — | q.s. | — |
| Purified water | Vehicle | q.s. | q.s. | q.s. |

TABLE 10
Zeta-Potential Profile of Suspension Compositions

| | | | Microcrystalline |
|---|---|---|---|
| | | | cellulose and |
| Suspending | | | carboxymethyl |
| agent | Xanthan Gum | Carbomer | cellulose sodium |
| Concentration | 0.3% | 0.20% | 1.25% |
| Examples | Ex. 22 | Ex. 23 | Ex. 24 |
| pH | 4.83 | 5.04 | 4.81 |
| Viscosity (cPs) | 523.89 | 132.37 | 425.91 |
| Zeta Potential | 21.08 | 27.06 | 57 |
| (mV) | | | |

Results: The data reported in Table 10 demonstrate that the eslicarbazepine suspension compositions with only microcrystalline cellulose and carboxymethyl cellulose sodium as a suspending agent have stable pH, viscosity, and zeta-potential profile as compared to the suspension compositions having other suspending agents such as xanthan gum and carbomers. There was a clear phase-separation observed for suspension compositions other than microcrystalline cellulose and carboxymethyl cellulose sodium as a suspending agent which clearly indicates failure in successful suspension formulation development with other suspending agents. The suspension compositions with only microcrystalline cellulose and carboxymethyl cellulose sodium as a suspending agent were free of any phase separation, a significant drop in pH, and viscosity.

### Examples 25-29—Ready to Use Suspension of Eslicarbazepine Acetate

Samples from each batch were analysed for preservative efficacy according to USP 32, <51>. The testing was performed promptly on the initial samples from each batch. Preservative concentration (100%) calculation is considered with methyl paraben used as 0.2% and propyl paraben as 0.05% w/v. The methyl paraben and propyl paraben are used at a constant ratio of 1:0.25. Based on the results provided in Table No. 11 it can therefore be concluded that the suspension compositions as per the present invention exhibited excellent preservative efficacy.

| | | Quantity % w/v | | | | |
|---|---|---|---|---|---|---|
| Ingredients | Function | Ex. 25 | Ex. 26 | Ex. 27 | Ex. 28 | Ex. 29 |
| Preservative Concentration (%) | | 0% | 25% | 50% | 100% | 150% |
| Eslicarbazepine Acetate | Drug | 10 | 10 | 10 | 10 | 10 |
| Glycerine | Vehicle | 10 | 10 | 1 | 10 | 10 |
| Polysorbate 80 | Surfactant | 0.04 | 0.04 | 0.04 | 0.04 | 0.04 |
| Methyl paraben | Preservative | — | 0.05 | 0.1 | 0.2 | 0.3 |
| Propyl paraben | | — | 0.013 | 0.025 | 0.05 | 0.075 |
| Sorbitol | Sweetener | 10 | 10 | 10 | 10 | 10 |
| Thaumatin | | 0.2 | 0.2 | 0.2 | 0.2 | 0.2 |
| Microcrystalline Cellulose | Suspending | 1.25 | 1.25 | 1.25 | 1.25 | 1.25 |
| and Carboxymethyl | agent | | | | | |
| cellulose sodium | | | | | | |
| Citric Acid | Buffering | 0.095 | 0.095 | 0.095 | 0.095 | 0.095 |
| Trisodium Citrate | agent | 0.15 | 0.15 | 0.15 | 0.15 | 0.15 |
| Flavoring Agents | Flavors | 0.32 | 0.32 | 0.32 | 0.32 | 0.32 |
| Purified Water | Vehicle | q.s. | q.s. | q.s. | q.s. | q.s. |

Results: The data reported in Table 11 demonstrate that the compositions containing less than 25% of preservative level failed to prevent the microbial growth in suspension compositions as per regulatory requirements.

TABLE 11
Preservative efficacy test:

| Example No. | Organism | Result |
|---|---|---|
| Ex. 25 | E. coli | Not Complies |
| No | S. aureus | as microbial growth increased from the |
| preservative | P. aeruginosa | initial calculated count at 14 and 28 days |
| | C. albicans | |
| | A. niger | |
| Ex. 26 | E. coli | Not Complies |
| 25% | S. aureus | as microbial growth increased from the |
| Preservative | P. aeruginosa | initial calculated count at 14 and 28 days |
| | C. albicans | |
| | A. niger | |
| Ex. 27 | E. coli | Complies |
| 50% | S. aureus | as no increase in microbial growth from the |
| Preservative | P. aeruginosa | initial calculated count at 14 and 28 days |
| | C. albicans | |
| | A. niger | |
| Ex. 28E. coli | | Complies |
| 100% | S. aureus | as no increase in microbial growth from the |
| Preservative | P. aeruginosa | initial calculated count at 14 and 28 days |
| | C. albicans | |
| | A. niger | |
| Ex. 29 | E. coli | Complies |
| 150% | S. aureus | as no increase in microbial growth from the |
| Preservative | P. aeruginosa | initial calculated count at 14 and 28 days |
| | C. albicans | |
| | A. niger | |

### Manufacturing Procedure for Examples 14-29

Preparation of Phase-I: 1) Sweetener was added to half amount of purified water with stirring for a suitable time (10-30 minutes). 2) Suspending agent was added slowly under sifting through a suitable size mesh (such as #40) into the solution of step 1 with stirring for a suitable time (10-30 minutes). 3) Buffering agents were added to purified water in a separate vessel with stirring for a suitable time (10-30 minutes). 4) Buffer solution was added into suspension of step 2 with stirring for a suitable time (10-30 minutes). 5) Wetting agent was added into the suspension of step 4 with stirring for a suitable time (10-30 minutes). 6) Drug was added into the suspension of step 5 with stirring for a suitable time (10-30 minutes).

Preparation of Phase-II: 7) The vehicle was taken in a separate vessel and was heated at a suitable temperature (50°−80° C.±10° C.). 8) Preservatives were added with stirring for a suitable time (10-30 minutes). 9) The solution of step 8 was cooled at a suitable temperature such as 30° C.±5° C. 10) The solution of step 9 was added to the suspension of step 6 with stirring for a suitable time (10-30 minutes). 11) Sweetening agents and flavors were added in the suspension of step 10 with stirring for a suitable time (10-30 minutes). 12) Volume was adjusted with the remaining quantity of the water and was stirred for a suitable time (10-30 minutes). 13) The suspension of step 12 was homogenized for a suitable time (20-30 minutes).

### Example 30

The Eslicarbazepine acetate oral suspension formulation of Examples 8, 24, and 28 were evaluated for pharmacokinetic parameters in human subjects. The objective of the trial was to characterize the pharmacokinetic exposure and to assess the steady-state comparison of the test formulation (T) [Eslicarbazepine acetate oral suspension 100 mg/mL (8 mL) (dose of 800 mg)] with reference formulation (R) [APTIOM® (eslicarbazepine acetate tablets 800 mg of Sunovion Pharmaceuticals Inc.] in a healthy adult, human subjects, under fasting conditions. The safety of the study subjects was also monitored and evaluated.

Study Design: The study design was a randomized, open-label, balanced, two-treatment, two-sequence, two-period, single-dose, two-way crossover, oral bioequivalence study. The study was carried out in healthy adult, human subjects, under fasting conditions. Healthy human volunteers who were willing to participate in the study and fulfill the inclusion and exclusion criteria were selected for the study. Blood samples were taken at determined intervals. Pharmacokinetic parameters used to evaluate and compare the relative bioavailability, and therefore bioequivalence, of the two formulations after a single oral dose administration under fasting conditions were Cₘₐₓ, AUC₀₋ₜ, AUC_{0-inf}. Bioequivalence was determined using the 90% confidence interval for the exponential of the difference between the tablet (R) and the suspension (T). The test met the 80-125% confidence interval limits with a statistical power of at least 80%.

TABLE 12
Pharmacokinetic Parameters of Examples 8, 24, and 28 in Humans

| | Cₘₐₓ | AUC₀₋ₜ | AUC_{0-inf} |
|---|---|---|---|
| Parameter | [ng/mL] | [ng * hr/mL] | [ng * hr/mL] |
| Geometric Mean (T) | 16649.4 | 318128.3 | 337953.6 |
| Geometric Mean (R) | 16297.3 | 310262.5 | 325246.7 |
| Point Estimate (T/R Ratio) (%) | 102.13 | 102.38 | 103.80 |

Conclusion: The results indicate that the suspension composition as per the present invention is bioequivalent to APTIOM® tablets (eslicarbazepine acetate oral tablets 800 mg). There were no serious adverse events reported during any of the studies.

What is claimed:

1. An immediate release oral ready to use pharmaceutical suspension composition comprising:
a) eslicarbazepine acetate present at from about 0.1% to about 40% w/v;
b) one or more suspending agents present at from about 0.01% to about 10% w/v;
c) one or more surfactants present at from about 0.01% to about 7% w/v; and
d) a pharmaceutically acceptable liquid carrier present at from about 10% to about 95% w/v;
wherein pH of the suspension is from 3 to 6.5 and buffer concentration is from about 5 mM to about 25 mM and wherein the suspension is free of suspending agents selected from the group consisting of xanthan gum and carbomer.

2. The immediate release oral ready to use pharmaceutical suspension according to claim 1, wherein the suspension comprises one or more other pharmaceutically acceptable excipients selected from the group consisting of preservative, anticaking agent, pH adjusting agent, antifoaming agent, sweetening agent, and flavoring agent.

3. The immediate release oral ready to use pharmaceutical suspension according to claim 1, wherein the suspending agents are selected from the group consisting of cellulose derivatives and co-processed spray-dried forms of microcrystalline cellulose and carboxymethyl cellulose sodium; carbomers; gums; pectin; propylene glycol alginate; dextran; gelatin; polyethylene glycols; polyvinyl compounds; sugar alcohols; colloidal silica; maltodextrin; starch; and combinations thereof.

4. The immediate release oral ready to use pharmaceutical suspension according to claim 1, wherein surfactants are selected from the group consisting of sodium lauryl sulphate; cetrimide; polyethylene glycols; polyglycerin fatty acid; sorbitan fatty acid esters; polyoxyethylene sorbitan fatty acid esters; polyoxyethylene alkyl ethers; polyoxyethylene castor oil; polyoxyethylene-polyoxypropylene block copolymers; and combinations thereof.

5. The immediate release oral ready to use pharmaceutical suspension according to claim 2, wherein the preservative is selected from the group consisting of parabens and their salts; sorbic acid; sodium sorbate; potassium sorbate; calcium sorbate; benzoic acid; sodium benzoate; potassium benzoate; calcium benzoate; methyl hydroxybenzoate; ethyl para-hydroxybenzoate; sodium ethyl para-hydroxybenzoate; sodium metabisulphite; chlorhexidine; diazolidinyl urea; sodium citrate; butylated hydroxyl toluene; butylated hydroxyl anisole; tocopherol; ethylenediamine tetraacetic acid; propyl gallate; quaternary compounds; phenyl ethyl alcohol and combinations thereof.

6. The immediate release oral ready to use pharmaceutical suspension according to claim 2, wherein the pH adjusting agent is selected from the group consisting of citrate buffer; phosphate buffer; monosodium dibasic phosphate; gluconic acid; lactic acid; citric acid; trisodium citrate; acetic acid; maleic acid; tartaric acid; fumaric acid; sodium phosphate; sodium gluconate; sodium lactate; sodium citrate; sodium acetate potassium citrate; sodium bicarbonate; potassium bicarbonate; sodium dihydrogen phosphate and potassium dihydrogen phosphate; hydrochloric acid; sulfuric acid; phosphoric acid; sodium hydroxide; potassium hydroxide; sodium carbonate; sodium hydrogen carbonate; magnesium carbonate; calcium carbonate; magnesium oxide; ammonia; synthetic hydrotalcite; lysine; arginine; meglumine; and combinations thereof.

7. The immediate release oral ready to use pharmaceutical suspension according to claim 2, wherein the sweetening agent is thaumatin; sorbitol; and a combination thereof.

8. The immediate release oral ready to use pharmaceutical suspension according to claim 1, wherein the buffer concentration is from about 10 to about 20 mM.

9. The immediate release oral ready to use pharmaceutical suspension according to claim 1, wherein the viscosity of the suspension is from about 100 cps to 1000 cps.

10. The immediate release oral ready to use pharmaceutical suspension according to claim 1, wherein the suspension has a zeta potential value of +50 to +65 mV.

11. The immediate release oral ready to use pharmaceutical suspension according to claim 1, wherein the suspension has a sedimentation volume ratio of more than about 0.9 for at least 10 hours after the suspension is prepared.

12. The immediate release oral ready to use pharmaceutical suspension according to claim 1, wherein the suspension has preservative efficacy and the concentration of one or more preservatives is 25% or more.

13. The immediate release oral ready to use pharmaceutical suspension according to claim 12, wherein the preservative efficacy is evidenced by the reduction in viable cell count of Staphylococcus aureus (S. aureus); Escherichia coli; Pseudomonas aeruginosa; and fungi such as Candida albicans; and Aspergillus brasiliensis (niger).

14. The immediate release oral ready to use pharmaceutical suspension according to claim 1, wherein the eslicarbazepine acetate has a D90 particle size between 5 μm to 50 μm.

15. The immediate release oral ready to use pharmaceutical suspension according to claim 1, wherein the suspension is free of xanthan gum and polyoxyethylene stearate and exhibits more than 75% of drug release within 15 minutes, when placed in a dissolution vessel filled with 1000 ml of acetate buffer, pH 4.5 maintained at 37±0.5° C. and stirred at a paddle speed of 100 rpm using a USP Type II (paddle) apparatus.

16. An immediate release oral ready to use pharmaceutical suspension composition comprising:
a) from about 0.1% to about 20% w/v of eslicarbazepine acetate;
b) from about 0.01% to about 10% w/v of one or more suspending agents selected from the group consisting of cellulose derivatives; co-processed spray-dried forms of microcrystalline cellulose and carboxymethyl cellulose sodium; gelatin; polyethylene glycols; polyvinyl compounds;
sugar alcohols; colloidal silica; maltodextrin; starch; and combinations thereof;
c) from about 0.01% to about 7% w/v of one or more surfactants selected from the group consisting of sodium lauryl sulphate; polyethylene glycols; polyglycerin fatty acid; sorbitan fatty acid esters; polyoxyethylene sorbitan fatty acid esters; polyoxyethylene alkyl ethers; polyoxyethylene castor oil; polyoxyethylene-polyoxypropylene block copolymers; and combinations thereof;
d) from about 0.01% to about 15% w/v of one or more pH adjusting agents selected from the group consisting of citrate buffer; phosphate buffer; monosodium dibasic phosphate; citric acid; trisodium citrate; acetic acid; sodium phosphate; sodium citrate; sodium dihydrogen phosphate and potassium dihydrogen phosphate; hydrochloric acid; sodium hydroxide; and combinations thereof;
e) from about 0.001% to about 4% w/v of one or more preservatives selected from the group consisting of parabens and their salts; benzoic acid; sodium benzoate; potassium benzoate; methyl hydroxybenzoate; ethyl para-hydroxybenzoate; chlorhexidine; butylated hydroxyl toluene; butylated hydroxyl anisole; tocopherol; quaternary compounds; and combinations thereof;
f) from about 0.01% to about 70% w/v of one or more sweetening agents selected from the group consisting of thaumatin; sorbitol; and combinations thereof.
g) from about 0.01% to about 5% w/v of one or more flavoring agents; and
h) from about 10% to about 95% w/v of a pharmaceutically acceptable liquid carrier selected from the group consisting of water; glycerin; or a combination of water and glycerin;
wherein pH of the suspension is from 3 to 6.5, buffer concentration is from about 10 mM to about 20 mM, a zeta-potential value of +50 to +65 mV, and wherein the suspension is free of xanthan gum; carbomer; and polyoxyethylene stearate and comprises less than 1% of SLB-1 impurity.

17. The immediate release oral ready to use pharmaceutical suspension according to claim 16, wherein suspension when administered to a human is bioequivalent within the 80-125% confidence interval and with a statistical power of at least 80% to an 800 mg tablet of eslicarbazepine acetate in a bioavailability study in humans.

18. The immediate release oral ready to use pharmaceutical suspension according to claim 16, wherein the suspension exhibits a mean Cₘₐₓ of about 16649.4±2722.1 ng/mL, AUC₀₋ₜ of about 318128.3±55124.2 ng·hr/mL, and AUC_{0-inf} of about 337953.6±61304.2 ng·hr/mL.

19. An immediate release oral ready to use pharmaceutical suspension composition free of xanthan gum, carbomer, and polyoxyethylene stearate consisting of:
a) eslicarbazepine acetate present at from about 10 mg/ml to about 100 mg/ml;
b) one or more suspending agents selected from the group consisting of a mixture of microcrystalline cellulose and carboxymethyl cellulose sodium; propylene glycol alginate; starch; and combinations thereof present at from about 0.1 mg/ml to about 35 mg/ml;
c) one or more surfactants selected from the group consisting of sodium lauryl sulphate; sorbitan fatty acid esters, and polyoxyethylene sorbitan fatty acid esters, and combinations thereof present at from about 0.01 mg/ml to about 10 mg/ml;
d) one or more pH adjusting agents selected from the group consisting of citric acid; trisodium citrate; sodium citrate; hydrochloric acid; sodium hydroxide; and combinations thereof present at from about 0.01 mg/ml to about 10 mg/ml;
e) one or more preservatives selected from the group consisting of parabens and their salts; benzoic acid; sodium benzoate; and combinations thereof present at from about 0.001 mg/ml to about 10 mg/ml;
f) one or more sweetening agents selected from the group consisting of thaumatin; sorbitol; and a combination thereof present at from about 10 mg/ml to about 300 mg/ml;
g) one or more flavoring agents present at from about 0.001 mg/ml to about 10 mg/ml; and
h) a combination of water and glycerin as a pharmaceutically acceptable liquid carrier,
wherein pH of the suspension is from 3 to 6.5, buffer concentration is from about 5 mM to about 25 mM, and suspension composition has a zeta potential value of +50- to +65 mV.

20. The immediate release oral ready to use pharmaceutical suspension according to claim 19, wherein the suspension consists of:
a) about 100 mg/ml of eslicarbazepine acetate;
b) about 12.5 mg/ml of a mixture of microcrystalline cellulose and carboxymethyl cellulose sodium;
c) about 0.4 mg/ml of polysorbate 80;
d) about 2.4 mg/ml of citric acid and trisodium citrate;
e) about 2.5 mg/ml of methylparaben and propylparaben;
f) about 102 mg/ml of thaumatin and sorbitol;
g) about 3.2 mg/ml of one or more flavoring agents; and
h) a combination of water and glycerin as a pharmaceutically acceptable liquid carrier
